SEQUENCE LISTING (22 sequences)

SEQ ID NO: 1 - DNA, 527 bp, Mus musculus
  source 1..527: mol_type = genomic DNA
taatgagggccctgtaactcgagtcagagcatgtaattctcttcctaggttgctctccagctagacttttatgactttaagtacttcatgatcagtaaataataaattcaatttaccagatagaagttttgtcacttagaaacactcacttggaatatatatttcttaattggcatcagtggcacatttcccaggaagcataacaaaacggatcatttaatagatgcccaatctctccatccatctatctatcagtgcggtacatgatgttttgactttaatatggtaattatctcaggctgacttcagagcaggaagctgcctcccacacagcagtgcttaattactttagacttcagcttgccaagtgaaaaattgactgtttttttgcacattgcatggaatcgaaatgatttcattgtcatttcagctgagatacagcctgacatctgcatagaatcacggcaaagataatgcagaatttctctgaaattctaaaatggcctgaaaagctgccttattttatt

SEQ ID NO: 2 - DNA, 516 bp, Homo sapiens
  source 1..516: mol_type = genomic DNA
aggcagctttcagaccattttagaatttcagaggaattttgcattatctttcccatgattctatgctgatgtcaggctgtatttcaactgaaatgacaatgaaatcatttcaattccatgcaatgtgcaaaaaaacagtcaatttttcacttggcaagctgaagtctaaagtaattaagcactgctgtgtgggaggcagcttcctgctctgaagtcagcctgagataattaccatattaaagtcaaaacatcatgtgccgtgctgatagatagatggatggagagattgggcatctattaaatgatccattttgttatgcttcctgggaaatgtgccattgatgccaattaagaaatatatattccaagtgagtatttctaagtggaaaaacttctatctggtaaattgaatttattatttactgatcatgaagtacttaaagtcataaaatctagatagagagcaacctatgaggagaattacatgctctgtcttggtttacaaacccctcatta

SEQ ID NO: 3 - DNA, 403 bp, Mus musculus
  source 1..403: mol_type = genomic DNA
ctgtacccggtaggagtgcttttgccatcagtaagaggaagaatcctggcgatgtttccagggaaaggaaaggagaacagaaaaggtggggcctccaatcaggccagagagggtgaggcagagccatctccacttcctttgtgcgtctcctagaatcacccccctcccattccaatctcctgtggcttttacagataagggtttctctccccctttctcactctccatctctcccaggttcactggaggcctctgagggcttgagataggtttttctttgcataagccaaacttctacttaacccagtgagtgctcttatgcagaaggtacctttaagtgatcacctgactgaagcgaatgtgatgttgcttatccttttatcaaaacatggaccaattcccc

SEQ ID NO: 4 - DNA, 456 bp, Homo sapiens
  source 1..456: mol_type = genomic DNA
ggaaaatcagtgcaggtttccagaagaggatgtgacaactcacattttgttcaattgagttaaataagcacttatagagtgtctaccacatgcagaacactcactgaatcgagtatggatgatacaaagaaaaatctatcccaagccctcctgcctacactgcctgagcctcatgggaccctaaataagtttggtaaggaatgagtggtgagaaagggtgaggagaaacaagagcacacatcgtcttgatacgggatctaaatgggtgcatggttctaggagaggcacaagggtttatggaggtagagattcccagtgtcccagatttgtttcttcactctcaggtcaaagcaggccttatttaggaggcccagtcattcctcatttcccttccttggtaatatcactacgattctctctcctcccactggccaaagtgtttgattaagcctgcag

SEQ ID NO: 5 - DNA, 612 bp, synthetic construct
  misc_feature 1..612: note = Synthetic
  source 1..612: mol_type = other DNA
acagtggtctcagcattcggtcagctgtgaggaggccttgcgaggctccaggccctgcacttcccaccagcatttggcgtccttgtgtttaatttggaccatgcacacgtgtgacaccccagtgtggttgtggtcaccgattcatgatgacccctctttcagtgtttattgttctgtccgtgtcttgctctctgaagactcagtgagattcgcattggaatatttgcggttttattgaagagaagagcttggggtctttgacagacgtgtgttaggaacatttctccatctgcggttgccatttactttttaattgcatcttctgatgattgttttaaattttaatgacgtctaatttgcccagtttttcttctgtggttagtttgtcccaaacacatggtggggcactcctgatgtctgctttgagtcagtacattgcaggtttgatatgcatgtctcacactgtgccgtgtggctctattttctttaactggcacacagtccttctgacatcctactgtcatttcttggggaacgttatgcttctctctgagtaactgtggtcccttgtgagtaccagtgctggtgggcggtggccttcttcttcactcc

SEQ ID NO: 6 - DNA, 1296 bp, synthetic construct
  misc_feature 1..1296: note = Synthetic
  source 1..1296: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaagagcccttcccagatgtcaccttcacagtgaccatgcggagacgcatgggttattatctgatccaaatgtatatcccaagcttgcttatagtgattttgtcatggatctccttctggattaatatggacgccgctccagctagggtcggactgggcatcaccacagtgctgacaatgactactcagagctcaggcagccgagccagcttgcccaaggtttcttacgtgaaggccatcgatatctggatggctgtctgccttctgtttgtcttcagcgcactgctggaatacgccgctgtcaattttgtgtctcgacagcataaagagctgttgcggttcagaagaaaacgacgccaccacaaagaggatgaggcaggagaaggacgcttcaactttagcgcctatggtatgggacctgcttgcctccaggctaaagacggaatttccgtgaagggagccaacaatagcaacacaaccaacccaccccctgctccatctaagagcccggaggaaatgcgcaaactctttattcagagagcgaaaaagatcgacaaaatctcccggatcggattccccatggctttcctgattttcaacatgttttattggatcatctacaagattgtgcgaagggaggacgtacacaaccagtaa

SEQ ID NO: 7 - protein, 431 aa, synthetic construct
  REGION 1..431: note = Synthetic
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVTMRRRMGYYLIQMYIPSLLIVILSWISFWINMDAAPARVGLGITTVLTMTTQSSGSRASLPKVSYVKAIDIWMAVCLLFVFSALLEYAAVNFVSRQHKELLRFRRKRRHHKEDEAGEGRFNFSAYGMGPACLQAKDGISVKGANNSNTTNPPPAPSKSPEEMRKLFIQRAKKIDKISRIGFPMAFLIFNMFYWIIYKIVRREDVHNQ

SEQ ID NO: 8 - DNA, 1416 bp, synthetic construct
  misc_feature 1..1416: note = Synthetic
  source 1..1416: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaacccttccctgacgtaacattcacagttatcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcgggacgagatgcgcgaagtcgctcgcgactggctgcgagtgggctacgtccttgacaggctgctgtttcggatctacttgctggcggtgctggcttattccattactctggtgacactctggtccatatggcactacagttag

SEQ ID NO: 9 - protein, 471 aa, synthetic construct
  REGION 1..471: note = Synthetic
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVIIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKQDEMDEVAADWLRVGYVLDRLLFRIYLLAVLAYSITLVTLWSIWHYS

SEQ ID NO: 10 - DNA, 1416 bp, synthetic construct
  misc_feature 1..1416: note = Synthetic
  source 1..1416: mol_type = other DNA
atgcgctgttctccaggcggcgtgtggctcgccctggctgcttcccttctgcacgttagcctgcagggtgagttccagcgcaaactgtataaggagcttgttaagaattataaccccctggagcggccggtcgcaaatgattcccagccactgacagtgtacttcagcctctccttgctgcagatcatggacgtggatgaaaagaaccaggtgctgaccactaatatttggttgcagatgtcctggaccgatcactacttgcagtggaatgtgagcgaatacccaggtgtaaagactgtaagattccctgacggccaaatctggaaaccagatatcctgctgtacaacagcgcagacgaaaggtttgatgcaacatttcacaccaacgtgggagtcaattcttcaggccactgcctgtacctgccccctggaatcttcaagtcctcatgctatatcgacgtccgctggtttcccttcgacgtccagcactgcaaactcaaattcgggagctggagctacggcggatggagcctggatctgcaaatgcaggaggctgacatctctggttacatcccgaatggggagtgggaccttgtgggaatccccggtaaaagaagcgagcgattttatgaatgctgcaaggaacccttccctgacgtaacattcacagttatcatcagaagaaggccattgttctacgccgttagtttgttgctccccagtatttttctcatggtcgtggacatcgtgggattttgtctcccacctgatagcggggagagggtctcctttaagattaccttgttgctcggctattctgtatttctgatcatcgtgtccgatacccttcctgccacaatcggcactccgctgataggagtgtatttcgtcgtgtgtatggcactcctggtgataagtctggcggaaactatcttcattgtacggctggtacataagcaggacctgcaaagacccgtgccagactggttgcgacaccttgtgctggacagaattgcatggattctgtgtcttggcgagcaacctatggcccaccggccacctgcaacctttcaagccaacaagacagacgattgtagtgggtctgatctgttgcctgctatggggaatcactgctcccatgttgggggaccacaagatttggaaaagaccccacgggggcggggatcaccccttcctcctccccgagaagcctctctcgctgtccgggggctgctccaggaactgtcaagcatccgacattttctggagaagcaggacgagatggacgaagtcgctgccgactggctgcgagtgggctacgtccttgacaggctgctgtttcggatctacttgctggcggtgctggcttattccattactctggtgacactctggtccatatggcactacagttag

SEQ ID NO: 11 - protein, 471 aa, synthetic construct
  REGION 1..471: note = Synthetic
MRCSPGGVWLALAASLLHVSLQGEFQRKLYKELVKNYNPLERPVANDSQPLTVYFSLSLLQIMDVDEKNQVLTTNIWLQMSWTDHYLQWNVSEYPGVKTVRFPDGQIWKPDILLYNSADERFDATFHTNVGVNSSGHCLYLPPGIFKSSCYIDVRWFPFDVQHCKLKFGSWSYGGWSLDLQMQEADISGYIPNGEWDLVGIPGKRSERFYECCKEPFPDVTFTVIIRRRPLFYAVSLLLPSIFLMVVDIVGFCLPPDSGERVSFKITLLLGYSVFLIIVSDTLPATIGTPLIGVYFVVCMALLVISLAETIFIVRLVHKQDLQRPVPDWLRHLVLDRIAWILCLGEQPMAHRPPATFQANKTDDCSGSDLLPAMGNHCSHVGGPQDLEKTPRGRGSPLPPPREASLAVRGLLQELSSIRHFLEKRDEMREVARDWLRVGYVLDRLLFRIYLLAVLAYSITLVTLWSIWHYS

SEQ ID NO: 12 - DNA, 508 bp, synthetic construct
  misc_feature 1..508: note = Synthetic
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 13 - DNA, 589 bp, synthetic construct
  misc_feature 1..589: note = Synthetic
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 14 - DNA, 82 bp, synthetic construct
  misc_feature 1..82: note = Synthetic
  source 1..82: mol_type = other DNA
aacttgtttattgcagcttataatggttacaaataaagcaatagcatcacaaatttcacaaataaagcatttttttcactgc

SEQ ID NO: 15 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 16 - DNA, 141 bp, synthetic construct
  misc_feature 1..141: note = Synthetic
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 201 bp, Mus musculus
  source 1..201: mol_type = genomic DNA
gtcacttagaaacactcacttggaatatatatttcttaattggcatcagtggcacatttcccaggaagcataacaaaacggatcatttaatagatgcccaatctctccatccatctatctatcagtgcggtacatgatgttttgactttaatatggtaattatctcaggctgacttcagagcaggaagctgcctcccacac

SEQ ID NO: 18 - DNA, 201 bp, Homo sapiens
  source 1..201: mol_type = genomic DNA
gtgtgggaggcagcttcctgctctgaagtcagcctgagataattaccatattaaagtcaaaacatcatgtgccgtgctgatagatagatggatggagagattgggcatctattaaatgatccattttgttatgcttcctgggaaatgtgccattgatgccaattaagaaatatatattccaagtgagtatttctaagtgga

SEQ ID NO: 19 - DNA, 228 bp, Mus musculus
  source 1..228: mol_type = genomic DNA
aaggaaaggagaacagaaaaggtggggcctccaatcaggccagagagggtgaggcagagccatctccacttcctttgtgcgtctcctagaatcacccccctcccattccaatctcctgtggcttttacagataagggtttctctccccctttctcactctccatctctcccaggttcactggaggcctctgagggcttgagataggtttttctttgcataagccaaac

SEQ ID NO: 20 - DNA, 276 bp, Homo sapiens
  source 1..276: mol_type = genomic DNA
tggatgatacaaagaaaaatctatcccaagccctcctgcctacactgcctgagcctcatgggaccctaaataagtttggtaaggaatgagtggtgagaaagggtgaggagaaacaagagcacacatcgtcttgatacgggatctaaatgggtgcatggttctaggagaggcacaagggtttatggaggtagagattcccagtgtcccagatttgtttcttcactctcaggtcaaagcaggccttatttaggaggcccagtcattcctcatttccct

SEQ ID NO: 21 - DNA, 179 bp, Mus musculus
  source 1..179: mol_type = genomic DNA
ttggaatatttgcggttttattgaagagaagagcttggggtctttgacagacgtgtgttaggaacatttctccatctgcggttgccatttactttttaattgcatcttctgatgattgttttaaattttaatgacgtctaatttgcccagtttttcttctgtggttagtttgtcccaaa

SEQ ID NO: 22 - DNA, 26185 bp, Homo sapiens
  source 1..26185: mol_type = genomic DNA
atcagagttgctttcttgggtctggatttgtgtgcttgcatggctctgctggcctgtggaaatatgtggagctattttcccatcctggaggccaggatcttacaggaaggcaggggcaggaaggttgggcatcatcttttcatcccaaggtggttaaaaggtgaagtcaatgcactatcaggatctcctccaatttctgtcttgtaaaatatttgagtcatgaatcacggagcaggttatcaagggacgctgggttctgtcttgaaatcatctcttacatatgaagaacaagatcagattaactttgtgtggacttatttaaggaaacactgattcaatgcaagggcccgaagagaaagccacagtgctggtcggctcagagacagaggctgcctgagtgtagagaatgtttaaatgttggattctgacctcaggcgaaattttgaatttgcagtctgtactgttctgtctgtacagtgcaaaatattttcttatttcaattgagaaactctctcatgaaagatattctaattattagcatatttacattttccttacgatattaaaacctgcacagaaatattcagaactattatcaaaatatgatcatttcccgtcaaaagtagaaaatactttttgcctctttcgggaacacatgataaaaataaactgatgactgagaagcttaagctctttaagaaatgtggatttgtaaagagatgatccaaaagggagaaacaagcaaaggaggattttgagagcttcaggaggtggccacacagtgaggttagctttgtatcctaggggattcttaccaagaaaaaccttgtattgcaaggcctggaaacaatgtaatatgtaaccaaatggtaacagtaacagctgggaaagagagaaaggctggctttggcagatgaaccaggagaattgtgttcctttttatcccctggggacactaaattcccaaacgcatattatttagttatttttagatatgcagcctgcagggaaaccgctgagaacattcccgtgtaaatcctcattcagctttctgaagtcagcattgtctgatgagtacctgttacagaggagggtttggggtggctgcgctgtgttccttcataggaaccgttgacatgtgtttaggggtgtctgggcactgtgacctcgaggcagtgtccgctgagagtgccagaggatggatggcgccttgaagttactccgttttctctgccatccaatgaattgcttgtaaatttcctgccacattactattgcatttcccaaaaataagataaacatcctcaaaatgtcataagctatatttttaatttgaatgttggtctgtatttctgtcatatgcaaaattgaaaaaaaaagaaaacaaaaacaaataatttcaagttctaagatgtttcccctgaggagttttactgggcatgtgtatggctatgggaattccacgttaattctaaacagaatattcaactgccagtaaactctgagacgaactatttttatttctcgagttggtattctaatgaagagggggaagtggatccatatagtggcttcagtctgttggaagagaaagaactacactttctatttgtaatatgcatctttcagcatcatataaacatttaaacttggtcacacattagttagtagattttcaatactctaccaaaaggtagttcctcgccattctcctctataaatcagaatcaagctgaaatcagggtttttagttatctttggttgtgtaatgtcttaagattttaaaaacgtttccttttttacctaattcacttcagtaccccaacatgggtatgtatgttatggctatgatgacatttttgaggtgaagagactaatatattgggttttaaggtcttatatacatgtgcaaccccttagcgccagaagcagaaactaatcttttgtttctgagtccagggtctgagtgtttaggtgacagtgtcccctcagggtactagacagcttacagaatttaagtagaacatgccgaagcacggcaaattagtcaaaatggagcaaaaatatgtgcataacttaatttgtgatggccatttaagagtccaaaataatttcatttcactccttattttcctcttttaggttccgtaaatgaaaagttaccccaacgcgaaggcacaggaaaaacaggtaactatcttagaataaatgtctatgacgatatgtaacatctaacttatcccgtgaaataacttctcaagttaaagttgaaacatccactagtgatgatgatgagagccgaaccagtttgttacttctgcattcattcagcaatgccattcactcacgatcaccagctctgttgggcctgtgctccgctggggtcgctggtgttggacctgtgctccgaggcttgtttcgggaccaacggcagatgctgttaggaccccctggaacctcccagtctgtgactgtggagagggctgaggctcagtattttggctccctgtgggattctaaaccattgcagttattgaggaccctgataaagatgcctcaactcctccctacaaggaggaagaggaacagtttttctacctacagcaatccctgcctctgagactggtacaaacaagagctgaacagaacccttaaccttaaacatcatgtcgcagcctgcaggtcctgtttcggggtctgcggggtctgtcacatgcccaccatgacccccctgggaaagtctccatcctcttctctgcccttgcctcggccaacacccttcagtaccaggacacatttctgcttttctcttacatcagcctttaaagtcagacgttgttttcaagatgagaagaaatactttagtaccaaaattttaggtacttttcttttctagtgtaggttacagtgtaatgaaccagtaacaaacaaaaataatataaacacagaccaggccgaggcaggcagatcacctgaggtcaggagtttgagaccagcctggccaaaatggtgaaacgccatctctactaaaaagacaaaaattaaccaggcatggtggtgcatgcctgtaatcccaggtagtcaggaggttgaggcaggagaattgcttgagcccaggagatggaggttgcagtgagccgagatcacgccactgcactccagcctgggcaacagagtgagattctgtctcaaaaacaaaaacaaaataataaaaataaaaatcggagttttattcaaccaatcccttctgcatgtgtggaaactgtggtggggcccatccctgccctggaagatcctttgcaaaaagagttctgtattactaggacactgacattttggagggacaagctggcatgaaagtaaatgtcaggctttctgtccagagggtgcctaccaaggatgaggcaggactggcttcctggaaggggaaatatgtaacaggtgtgagctggaacagaagaagcctcacagtcaaggaagatagctcattttccagccgaaaaatgatgacaggcataatggggacactgggttgggggagggtttatgtttggaaaggaaggggtaaagttgcatagagcaatagtgtgagctgcgtgaactgagtcgagaagataatggtgtgggtatgtcgacatttgtagagaaaggaagggacaatctcatgattaggaaaattaatcaaatgtggaaagggaggaggagatggaaggatcatcgtctttcagagggcatttcttaatcctgtttattatgatatttattgtacccactgtaaaaaaaaaaaatctagaggaattagtacatgggcatgaaagtgagttaaggaaagaacacaccccccactttaggccggaatacgcagaggggatacctggattaagcaacagagctggtttccagacacaaataagacacttcacacactctgatagagggaaagtaaaaaatgcacagctcttcgagatttttgttttatttccttaaggctgacttcagcagccaccaatggccttgtttgttgggacagagccaccccgtctgtgagggacgggggcatggagtagggcctatgatgaccgtgctgaggagggaagcatggacagggcaggaaggagagcggcctctggtgcccagaatcagggctgggaggaggaacggccagtgggcccattactcaccctgtcctcaggccacacctgccctccctgcctgccattcaggctgagctttcagactggtctggccccagtgactggggaatgggctggtggtcccagagtggtgacttggtgaggcctcagcaaaaacacacaacagtaataatgtatttgattgtcctcattccaaaaaatttcaaatatgttcttttaagactttttgtatgttcttgtgtttaaaatatgtttcccattttgttttattttcttggaagaagattgtaaaatattcaaccacagaatattcctagtttattgttattatcagtgttagacttggatatttcaaataactgatgttacatttttgtcagtattttgatagctagcaaactgtttttgagagattgctcaatgttaaatttttttgaaaatctgttttgattttccccctaaaaagtcttataagaaactttattctttagaccactgggaagttcatgacttagttatctacgtaagccttgtatctggccctcatctttgccactttttcctatatttcaatccaaaagttatttaacaaaatgatataagcaagcttcagtgaaaaaaaaaaaaaaaaaaaaaaaaaaggatgatcctgagagcaccacagtgccttgtgattgcctcctgggtggggccctgagtgtctggagaaggactcctgactcacaggctccaccatcctgccaggtctctgagccatttaaaacctctttctttaccagggggcccaatgtgtgtgtgtcaattaattaaatacagattcttcacttggaaaatgcgagtctatgggtggtgtaggggcctgtgacaccccagtccgccctttcctccattctccgatcactttcgccctgtcccctccaccccgtgccccactcacatccttacttagattatgccatggcagcagtggagagagggagggccagaggtgtagaaaggggtccaggattctccaacccttgcctttctcttggggaaaacagaggatgtgttaagggcacagggctctgctcaaccccaccccagtccctgccaagccctcttttgtaacctccagtggcctgaatttatgacttctctttcccagggctagactttctttactctaattattttgattccttcctcatctgccctatggcctgtgattggctctgatagtttagaactgatcggatattttccacgtgaagttaggtcagtgtctagcctcgagcattttaagtagcggctcactcaagactggtatagtcattccagccgcagctgcaggcagcatcgcgttgggtctgtgtggggttgctgttgtggttgatctgtggggactgagtgcaagtcctcagcatccacccgtatgcacacttacttaacttactctcataattgtagtctccttgcaaatcttttccgttctgaattcagatgatgccgcagctccagcgttggagactcagcctcaaggagatgaagaaggtgagccggggtggggattgcacagtctgggttggggaggagtgcgagggacggtatggagctgggaagtggagcgagtcatcttgttagagctccgttcagtatggagtgtaacatgccagacacagacagaaaagaccacgtgatcttacttatatgtggaatctctttaaaaaaaacaaacaaacaaaactccaatagtgaggagaggaaaacagcaagccgaaaggtggtcacgaagggtagggtaggatggaggaaacagagggatgaggccagaggctgcgaagctgcagttacgtgggatggacaagtccacacatccggcgtacagcacgaggaatctagttagtgatgaaatgacaatggatggcataccacacatccagcacatagcatgaggaatatacttaatgatgaaacggcaacggatggcatagtaaacatttcctagccacacgcccctgaagtaactgtgagatgctggggatgtgaattgcttggctgtagaaataatttcactgggtaaatgtgtatctaaacatcatgatgttagacagggataaaagagcaaactcaagcagaaacggattgtgtgtggccgtgtccagccttatgagggcacccactgtttttcctgatgtctttggagcgagggattttcagatattgccatggtgcattctcatgagggtttcgttaaatacacatttctaagttgaaattttacagcttggataatataaaatgtatggccattttccctgtttacatttcacgtctttacaagtggtcataacaaggagagatttcctgtttttacctttaatacattgtcttaggtatttaccagtgtataaaaattatcaggtcaaagggaaccggggtttaagggcaaagtgtaaggagtacacagtataaattaacaaaaccttaaaatgtagaactgcattatgctttgcaaataataataatgactgatgctgcgtggtcctaagcgctacacaatgtgtgatctgctgccatgagtccctggaccttctgaatgcccttccgcagatgagagccctgtggcaattgacccaagtccatctgggtgtaaatggcaactggcattcaaacccagatgtcttcttcagggtctgtgctctcaagcactaacggtgacctgcactacattccttttttttttttttttttgtgagacagagttttgcttttgttgcccaggcttctggagtacaatgatgcgatctcagctcactgcaacctccgtctcctgggctcaagcgattctcctgcctcagcctcaggagtagctgggactacaggcacttgccaccacccctggctattttttttttttttgtattattagtagagacagggtttcaccatgttggccaggctggtcttgaacacccgacctcaggtgattcacctgccttggcctcccaaagcgctgggattaacaggtgtgagccaccatgcccggcccattcaattttatttgttaaacaagattactacaaaaggggtgttcctgaaaagtttcaggcaaaagttatacttcacatattgcattttataatgaatctttcaactgagtgaaaacgttgtatgttatgtgaatagccattttaactacacaagaatatatttttgtacactgggtatttttctaaaatctgattctttcaaagtgaattctgtctagacccaaagagcttttaactatacttttggatagagagaagaatgccaaaaatattttaaatgtatattatgatttcctttcgtctgaggcatataatcagtgtaactatttctttacaaatgtacaatgtgtgttatgttgatttattttggctgttggactacttgctgcactgcgtaactaaggaacattgtgctttcgtgatgaattaaagatgcttgtcatttcattttggttcatagatattgcatcacgttaccctaccctttggactgaacaggttaaaagtcggcagaacaaaaccaataagaattcaggtaagatgctgcctgatgtcactttgtaaggagtgatttaatagatctagaaaatgatggggtttttttttggagttaagtgtattgtacagttgacccttgaacaacatgggggtgaggggtgcagacctggcacaaacatctgaggataactttcaactccccccaaaataataacctcttgtagactggcagccttacccataatacagttagttaacacatagtttgtatgttatatgtattatatactgtattcttacaaaaatagagaaaagaaaatttattaaaaatcataaggaatagaaaatatatttattattgattacatggaagtggatcatcataaaggtcttcgttctcctcatcgtcttcacgttgagtgggctgaggaggaggaagaggaggaggggctggccttgctgtgacagaggcaaaggataaatccttgtaacagtggacccatgacatcaaaactctgttgttcaggggtcagctgtaaatattctcacttggttactcagtcatgaaaataattcttattatttgatcaccggcatatatttttaaattcggcattttaaaattaaattttctaaattctttaacagatttctctgtggatgtcaaactatagctttagttaataagtaaataattataaatttgcttttgacctaatttcagagatgttaaaacactttgctaatttagtttaaaatcctccaaattgtgtgcattttgtttgctataaagcagtgtttgagagaactttcaccttttaaagctgactattttgacattttgaagaccagtaatgggttgcttcatctttggttaatgattttgactgttttcatggaagcgggtttgttctgtaattttcatttaggctctcagttgattgcttgtgtgtttctcattgccttttcaaataagaatttcacagattcttactgaagggaagtagctctgtaggtttaaagtagttcagctgctgaggaaatacggagttagtaacagcttgaattttctgcgatcagctttgggtgaaaaaattgcaaacattcacagaggctgcttatgcattaaaactttatctgtggttatgcatccatatattcttctgcttacaaaaaaaatcaaaaggtatattaaagttcaggagactaagagtggttcgattcccccttcacccagctcttacacacccaacctgaaaaggacccttgagggaagtgcctgagtccccttaagccagcagccacctcagaaccccatggcgagtctatggtaggcccgtccactgatttccacctgcaaagctagcgcggtgcaatgccagatgagctggcacaggtccaatgtgtgatctctgccccatgatgctctgaaatcactgcctccagccccggagtcttagctgtgtggcatcttcttgttactttacccttctgggcggagatgaggaagggcaaaccccaagggccccccaccgtctacaggcacacatggcagaaacatttaatacctatttataacatttttctcagaaatgcttgttatagatgagtttttcctattatatggagatattaatctctttacatgtctcatgaacttttgacactgggttttgttcaattttgtcaccccatttaaaaaagttattttgatttatttctgtttcatacaaatttgtatatgagagattaattaaaacactgtatgtttcaggtttactggttgatatggttgggcgttgtgtccccacccaaatcttatttagaattgtaatccccataatctgcacatgtcaatggagagaccaggtggagataattgaatcatgagggcggttccccccatgctgttctcatgatactgagtgagttctcacgagatctggtggttttatactgggctctaaccccttcgctgggcacttctccttcctgctggcttgtgatgaaggtgccttgcttcccttcaccttctgccatgatggtaagtttcctgaggcctccccagcaatgctgaaccgtgagtccattaaacctctttcgtttataaattgcccagtcttggggagttctttatagcaggatgaaaatggactaatacattggtagatcttgaaaaccattgaagaataaatatgttagcacctaaacttaatttgacattcttgtaaatgattttactggaaatatactttattttatttatttatttatttatttatttatttatttatttatttattttgagagaaagtcttgctctgtcacccaggctggcgagcagcagcacaatcttggctcactgtaacctccgcctcccaggttcaagcgattctcctgcctcagcctcctgagtagctgggactacaggcacccaccaccacacccagctaatttttgtatttctagtagagacagggtttcaccatgttagccaggcttgtctcaaactcctgacctcaagtgatctgcctgcctcagcctcccaaagtgctgagattatgggcgtgagccaccatgccaggctaagaaatataaatgtacatttttttaagccaatgtaaccatttcaggcttcagcagattaagctccatgtaaacctgtggaagaagagcatttctgtgtctcacctctcacttgccacttgcagatttccaagacttcaaatgacctgccattataattaaccagtaaaaactgagcaggcttttggtatttagtgggactgaaaataatgttttaggaaattccagtttacagaagttaccttacactataatttggtttaagtcagccgagatgagtggagcacgaaaagatggcattgagttgatttacagacacggggcatgaagctactggactatgtttgtgtttaataccagttttctagctacatgttctttctgaggggctgaagaaacacctagttggtattttaggacattaaaaataatatttaatttattaagttttaacaatgaaatgacaatcataggatatatataatgaccactcttattgaaatttcaaatatcagaagagtgtcatttatacttaattttaatttttaaaatatacttttacttgtaatcatcttcggtagacatagacacatttatttgagaaaatgtaagtaatttttgataataatttgggtatttaaaaaaatttatctagcttgtcagtaccttttcctgtttttttattaaaaaggccaaactgaaaatgcattgctttttgtttctctgatgtttctttatatcagataaaagtagtatttttccccatgaatttgtatgaaaataatgtgacttttgttcccgcagacatagaatatggttgccatgcatccaaatgcccatgtgcacactagcaagatgtggtcacatatgctttggagcatataatgatagtagtcttttaaaagggactatttttacaaaggaaaataaaatacttctctgaaaatttgcacatggtagctacacaataaaatcatgcctcctgtggggtttgaaccaaattgggatctgtaagtcaggcagaattccttcatacccaggccagactggatttagattggcaaagacccccccacccccccaccagcctgttgctcactccaggccccagcagagagcccaccgagggtggcccctcactgtcctccgagatgctctgcaggaggtgagtgacggcacttgaaaatttgcacatagtagctacacaataaatatgcattaaatgaataaagatttgtaccagcatatgttccacgggccctactgtagcatcattactctttttaaaacaagctgaatgaaacaaacaaaataaccaaattaatacataactggaaaagagagaatctaggcacccctagaggttgcctattagacttatattgatttaaatttaataaatatataagtcaaacattctcttggctcatttagtaagaaaatcatcactttcatcatgtgaatcatatacattaaataaacgagtaaaatctgaagttgcctgttacttaaaagtgcattactcagtttttcccatcaatataaaattgtgttacttcttttactgtctcaaatcatctttgttttattactcttttcatagaagatacataaactagttacaagattttatcagataagacattttgtaagatacagtcttttaattatgtaagatttaaacaaatgtaaatcgaattagtactcttccctactggcatattaagtactttctagatttctgcctgatttcctttaaaaccaatctaaaactgaagaggagactgcaacttcttttcttacttgagccagctactaacatcttcacaaaataacaaacactcttcatgaattaccaacaccaaggtgcttagggtttcatgacccattcataataaaacacatcccctctagacagaaaagcatatgatcgtggcatgtcaaagccaaggagggctctatctctggggcgaggaggcatccaggctcacgggacggtttctgtatcacggttgccatttgccaccatttgccatagtgacgtgacctatgggaaagagcactcagaaataatgggaagatggagaacatgtggaaggccagctgtctcacattttggtaggaggattattccatgggacgagtcgattaaaacttgcttttgaactgcgtgtgctgtgcatggtgccccttccccaccaggtatcagcgtttttcacgttttggtaggaagattattccatgggacgagtcggttaaaactcgcgtttgaactgcatgtgcacggtgccccttccccaccaggtatcagcttttttcacattttggtaggaggattattccatgggacgagtcggttaaaactcacctttgaactgcgtgtgctgtgcacggtgccccttccccaccaggtatccgcatctgtcacgtacagtgaggctgccatcatgttgttttcaggtgccatagtctcccacaagacaggaggaccagataagccccatcatgtgatacttactctatgcagatttcttaaagtcatgctgaaaaacttgtctcgatttccatcaattaattaacaccttggggtgtcctactactattatacagagcactgaagagaacatgaaagaaagaagaaataggtgcaggagctcaagaagcttctgttaagcttctgcaggaacttttttacacttaggaagttagaagcattctgctgggctcctgggctcctgggctcctgggcagcctgcccctggaaggggtgggggtggagaggaggggcacggaggccccccgtctcaggcctgcacaggccgttctgggactgcgtgtcctggaagacctttcctgattatcaggacacaaggccagacgtgggaccacacacagtcctcggagctggcagccagactttgtggctggcagttaaaggcaatttggtgttcgtggcccactggcaaggtgaatttggttgctttccttaacaccgtgtgcccaattcctgatggagccatgctgccacagggctttgccacgtggggagatggtcaggtcggctgcccaactacattctgtgttttggggacctcatttggttctgaaatgggtctcactttgcccaagatgccgggggcatctgctgctgggtcccctctcaccacagggcgtgtgctccctcgggcacgagttctctctggcatttttgaaaggccttttactgtgatatcagtgaaaatgaaagctgaagcagttcctgataaagggggaaagtggatttcgccaagtgggcctgactggtcccagcatctgcacagcgctcagcgctcgcctgcaagcgcagctcagggcttccctggggccagctcccggctcctctcggcctcgccctcaagtcgggactttccttcttcggtaacatgtctccaccgggcctgcaatcattagccagccgggatgagactaagtgcttaaaggctatttcatgttggagatttaaaaaagcacatattgcaaaagcggttcaacgcgcggagaaattcacccactcttgtctccacccgtcaaatatccggggtcctgtgacctggggtcctggggttggctggttgcgaagcagagtgacgaggaagacggaagagctctgttagcacgcagctcccaccatcaagcttcccaccaggggccaagcagggaagcccttaccttcccgtcgcctacacagagaggctgggtgctggctcagccagcccagcgagctccttctccctctgccagcaccctgctccccgtgggatttgggatggacgtatggcgtccacggccgcatgttacaaggtcccactcatagcatgcactgtggaacctaaatcatgcctcttgcgggatttgaaccaaattgggatctgtagctcaggccaaattccttcacacccaggccagattggattcagatgggcaaagacctcccagcagcctgcagcggggagtggccgcccggacacaccttcgctttagaggaagggtagaggaccgtcgagcttgatcctctacgccctgagcacctgtttctgttgacccctctcggccctgagcccttgtctcctgcgcggtcctcctaaggccgaggcagcaccgggcagtcccttaccagcagggtgggcggctgctggatgggtcggggccagacaccgctgggggcactgcgtggacgaggtggacgctgaagtcagggcctctgagcgacaccatcctcgccgtggcggggtccaggcaggcctggggcctcgggccatgcgggtctcagcccgaggcgtcgtcttcactgttgaggggaccccctccacgtccaggccttcccctccccgcacggggaagctgaagtgttcccgggggtccgcggctgtgcgggagggaaggagggactcaggtgggggtcccagctccgagggcgtccgtggcctctcccggctggggtggggctgcccacaggactctccccacgcccatactcgggaaccaggggaagctgcgggctcctttcctccagtgtccttgttgcccacatggggtgcgggagcagccttgggatgattagcttttaaggggagtccggctctgcaccctcggtccccagcagcccaccctgcggcccccgcacctccccacgcgggaagctgttcctcgtggcctcgcagactgtgcgggccctgggcactgatgcctcatctcctttatttcacatcccacgcgggcgctggaggcgtctgtttcctgcctcctccaaagctctcccaaaagcccagggctgtttgaggcccccaaaatgcgccttgggatcttttttttttaaattatactttaagttttagggtacatgtgcacaacgtgcatgtttgttacatatgtatacatgtgccatgttgtgctgcacccattaactcgtcatttaacattaggtatatctcctaatgctatccctcccccctccccccaccccacaacaggccccgctgtgtgatattccccttcctgtgtccatgtgttctcattgttctgttaggttttttttttgtgagctggagagaacgtagaggcctccagttttgtcatctgtgaaattcctcctgcttgggagaaaaatgccttcctccctaacccattaaaaacagttctgttcaatgtccaggaagagtccttgatgttcaggacatcccgcagttcatggagcccctcagtggaaatacttctttgctttatttagggatgataatttttgttatatcggacatattaaaattagtgccaaaagataattatcttttgattctgctttaaatcatgctaaaatgcattccctatgggaaaacgatacaaattatattttccttttttatttctaagtttttcgtgtttattctgtttttcaaatatgacattacacaaacatcaagacaccaattagatttgtaagttattaatttttggtcatttgaagagattaatgctaaattaaagcaaataccacctcataagtgtagactgtaaaagtgtattgtttaaaaacattaattaacttaccaaaaatatgcataactaaatttatccaattttcataattataaaagcttttaatttcaatcatatttgattctcagatttcaatattcaatcacactgtggttaagtaatcagaatgaagcttttcaacctaagtacagaaaatagtaatacaagcaaaccctatctgagtggtttggaaacacacaccaacacgtgtacacgcaggccttgagctctcgccatgaaagaaggggtatttgccttacttcgttttctaaatataaatgacatcaactcctccttgaactaatgaggtctgatgttgacatcactagttgctcatgggtctgctgttggcgtcactagttgctcatgggtctgatgttgacgtcactagttgctcatgggtctgatgttgacgtcaccagttgctcatgggtctgatgttgacctcatatgatgcttatgaggtaccaggcaaggcaccgtgtgttctaatgcatcatagaagctgattcttagaatagccatgtgaacttggagctgcttgtctcataggaagttcatttttcaaaatctcttaagtggctgaggccaggatttagcccagagttgtgatttccaacctcctcctgcggcccgcagattgacaggcacactcagttccagagctgctggcctctggggcatctctgaggctgacagtgtgggagaggccccgtggcacacacctgtgaatgctccctctcccagtgatactccaccaggcaagggactggcttcagaagcgatctcatggtaaacagcaccagcagaagctctctcaggctcacagatggggcctccaaagtgagaaccaggagagcggctgtgtctccaccctgaacctgcaggcagagccgcaaccttctcattgtcttatttaggagggatcatgtcaggcatggggaggagacgggagagtggttggtgtcatgtaccgtgaaggtgcattaggacactacctgccctaggaaccatcttttctgcacaagggaacacagtggctggagagatattaggtgtgctcactcctcctcctgcatgccatcagtccccctggaacctttcttcccatcgaagttattgttgctgctgctgggagcctccctggtccctgggaaggacagaaggggggcctgggctggttctcggccccccctgtgtgatcctgatcaatgcattttacctatctgggtttcagcatctcatctttaaaatggtaatggtaagacccacctcccggaaaagccccagagctcagtctggttggatgcaccagtgtccacttggtgtctaggaatggaattaacttccaggtgtgcacatcgagcttggtactggactcggcaaatagggattcagggcatctcttgaggctcttccctgaatgtgtgtgtgtttgtgcatgtgtgtgcacatgcgtatgtgtgggtgtgcgtgtgcgcatgtgtgtgtgtgcgtatgtgcatgtgtgtgcacgtgtgtatgtgcgcgtgtgcgcatgtgtgtgcatgtgtgtgcgtgtgtgcgtgtgcatgtgtgtgcacgtgtgtatgtgtgcttgtgcgcacgtgtgtgcgtgtgcatgcgtgtgcatgtgtgtgtacgtgtatgcgtgcacacatgcatgtttactgtaatagtaactagttttttgagaaatcaggtactggaaatcgtagtgacaggattaataggaccaagatttacatgttgttatgaaatttgtgtcgtcgtgattacagatttggcttgaagcataaactgagtgaaatatctctttttgccattcacacttaagaatagtaatttagggccaggtgtggtgtctcgtgcctgtaatcccagcactttgggaggccaaggtgggtggatcacctgaggtcaggagttcgagaccagcctggccaacatggtgaaacccggtctctactaaaaatacaaaattagctgggcatggtggcgggtgcctataatcccagatactcaggaggctgaggcaggagaatggcttgaacccagtaggcggaagttgcagtgagctgagattgtaccactgcactctagcctgagtgacagagtgagactctgtctcaaaaaaataaaaataaaaataaaaataaaataaaataaaataaaatagtaacatagtagaagagatttcactgtgattctgtttccatggtcttcccatcagttagcttaaagacccacagataattaatatggcccatgaataatatgaccctttgggagatgactcatgtagttgcactaagataagaattcatgcctgtgctcatctgccagccaaggtgcacccaccaacggcgtttccttcaaccgcaccttgttctatcaacagcaaccctctccctagctgtgagcaattaacgatttatctcgggtgactagtaaagaattttaccagcccccctaatatttttaccagtccatcctcctttgtgaaaggacaaagttcatacaattaatgaaatcatctttctctgtagtcacccttatcaagtgctgactgatgaggacttgtttgccaagcacgatgcattactatagttacaatcaaggcaatgactgcactgagagtttaaaaaatgttcatgcatttgctgaggaattttattcattccccaggaatggtgtggtgcatgaatgtgcacatgtgtatgtgtgtaaccacacatgtacttcataattattcttctctaaaaagacagctttatgtttttaatagaggcaacctttttccttagctctgttactttatatttatcatctttccccttttatgtacctgcagcctacatttgtgtctcagataaaatctagtatagacatggatctctctgcttttgcctgaaagtcacattagtaactaatgccagaatacgtcacgtcaggctattgtgtttcagatgaaagggcaaacgaagaagtcatttcaaaagcattgtaaagcaccttcttcagttatatgttgcgtctgcatcaaaacacagaacatttaagtgatggaagagtcatggttactgacaaacagtcttcaaggtcatatttattttttaaaactctttcaataactgaagaagaatgcgtcctctgtgcccacctttctggccctgagatgtgaggctctcactgcgttcttggaggaggtgtcattttcctgccctgagacacgaggctctcactgcattcttggaggaggtgtcattttcctgccctgagacacgaggctctcactgcattcttggaggaggtgtcattttcataccctgagacacgaggctctcactgcattcttggaggaggtgtcattttcataccctgagacacgaggctctcactgcgttcttggaggaggtgtcattttcctgccctgagacacgaggctctcactgcgttcttggaggaggtgtcattttcctgccctgagacacgaggctctcactgcattcttggaggaggtgtcattttcctgccctgagacacgaggctctcactgcattcttggaggaggtgtcattttcataccctgagacacgaggctctcactgcattcttggaggaggtgtcattttcataccctgagacacgaggctctcactgcgttcttggaggaggtgtcattttcctgccctgagacacgaggctctcactgcgttcttggaggaggtgtcattttcctgccctgagacacgaggctctcactgcattcttggaggaggtgtcattttcataccctgagacacgaggctctcactgcgttcttggaggaggtgtcattttcctgccctgagacacgaggctctcactgcattcttggaggaggtgtcattttcctgccctgagacacgaggctctcactgcattcttggaggaggtgtcattttcataccctgagacacgaggctctcactgcgttcttggaggaggtgtcattttcctgccctgagacacgaggctctcactgtgttctcggaggaggtgtcattttcctgccctgagacacgaggctctcactgcattcttggaggaggtgtcattttcctgccctgagacacgaggctctcactgcattcttggaggaggtgtcattttcataccctgagacacgaggctctcactgcgttcttggaggaggtgtcattttcctgccctgagacacgaggctctcactgcattcttggaggaggtgtcattttcataccctgagacacgaggctctcactgcgttcttggaggaggtgtcattttcctgccctgagacacgaggctctcactgtgttctcggaggaggtgtcattttcctgccctgagacacgaggctctcactgcattcttggaggaggtgtcattttcctgccctgagacacgaggctctcactgcattcttggaggaggtgtcattttcataccctgagacacgaggctctcactgcgttcttggaggaggtgtcattttcctgccctgagacacgaggctctcactgtgttctcggaggaggtgtcattttcctgccctgagacacaaggctctcactgcattcttggaggaggtgtcattttcataccctgagacacgaggctctcactgcattcttggaggaggtgtcattttcataccctgagacacaaggctctcactgcattcttggaggaggtgtcattttcctgccctgagacacaaggctctcactgcattcttggaggaggtgtcattttcataccctgagacacgaggctctcactgtgttctcggaggaggtgtcattttcctgccctgagacacaaggctctcactgcattcttggaggaggtgtcattttcataccctgagacacgaggctctcactgtgttctcggaggaggtgtcattttcctgccctgagacacaaggctctcactgcattcttggaggaggtgtcattttcctgccctgagacacgaggctctcactgtgttctcggaggaggtgtcattttcctgccctgagacacaaggctctcactgcattcttggaggaggtgtcattttcataccctgagacacgaggctctcactgtgttctcggaggaggtgtcattttcctgccctgagacacgaggctctgactgcgttttcggaggaggtgttattttcatgccgtcctcactttgggggtgaggaggtactgttttcctctttacatatgaaaaaatgcaaacgttcacccaaacaagaggcgagggctagatgacccgcctgagcttcagcttgccttgtttattgtcagagggtgcgtttctgatttacgcatccggggttatcaggtaggacggcttaaaatggtgaaagaaatgcactcacatttcagaggctcagcaggtcggaagcgcagtgttttatcacgtgatggtcctgccccgggagagcgttgcccgtggagctgttttcccggtgatcacgcagggatctggatccctccaggccgtattttcacggatgacagtttcaggagggggtagatcatcagaaggaggcagaatctttctcctaaaagctccagcctagaatcgaaccaaccctgttccctccatctcactggagcaaacacagtcacgtaggcatgctccccggaacgggggctgggagtggagactcgctgtgtgcctgccataattccgtctgcgtcgaggaagggggcggcttgggaagcagctgggcacctgtgccacagccgccacagactcagcctctgctgccattttaggaaaaagcaggtattcatcgcgatctctctgcatggtgtggatcctgtgtttaactcacactgatcacttgttcacttttcattttatggcattcctgagcttaagcacacacgtgcattcatacacaatatcgatgtatgtatgtatgtaagttttatgtgtgtgtgtgtatatatatgtgtgtatgtatataaagtttatatatgtgtatgtgtgtatatgtatgtgtgtatatgtgtacctgtgtatgtatataaagtttatatatatttgtgtgcatatatatatacacacacacgtacatagatacacagtcatacacacacacacatataaactttctttttgctaaaatattgtgtgtctgtttcttgtctccctgctatggtctaaatgtatgtgtgtccccaaattcctgtgttgaaatcctcacccccagggtgatggtgttaggggctggggcctttggaaggtgatgaggttgtgagggcaatgggattagtgccttatgaaaagaggcccaggagacccttgcccctcctgccacatgagctcaaccaagaaggcgccatctgtgaaccagggaatgggtccccagcagacactgaatctgccagaaccttgatcttgagcttctagcctctcaactgtgacaaatgaatgcctgttgataagccacggagcacagcagcacaggcccgcaccaggtaagactcctcccaaccggaggctcggtgtccgtagagcaagcagagctctgcgtctccggcttcaggggcctgcagggggcgggggcgggtgctcacctgcctcatctcacctctagtcctggttaaagcacaccctggagtagaacctctggggtgggtggggccttggagacgcaatttaagcagcttctcaggtaaattgttttatcttaaaatgtacagatcactgtgacacatggcaactattatcaataaggttttgtacatattttaatttttcccaaattacgtatgtgtataagacaaaacacgaagtttcttgaaggcatttgagttttagactcctttttgtattctagatacaagtccttaccagatttatgattcacacgtattttcttccactctgagccgtcctttcactctcttggtggggttcttcaaagaagagtgcctattctttgcaccccatctgcatgcctgtgtgtgtttatacaccactgcacaagtccccgtacagtacatgattctagggcactgtctaatcatgtatactgcatatgaaatataacacttcagactttatgacaaatccttgcagtaggttgtttgtctttaacttgtatgtatgtggatttaatgtattttcagaggtaaacattctagctaagctagaaagcctgaaaggaagtttcaggctttattatggatattgtagcacttctaatgcagagttagactttataaaatgtagtggatggcacattctgtcatactggaaatgcacagtgtttttaattttggtaaagtccagtttacctatcttttcttttgtggctatgcttttggtgccatgtctaaggaactagtgcctcacctgagtccccctttgtgagccagtgtcaagcccatgacaggaggctgatgacgggcagccgctgccacccagggtgacacagggctcactacccctaaggcagcgggtctcactgctgtgcaaacaatgcactctgatgcattctgctgacataaggttaacctggacctggaggggtaggacttgaagttaatcccactgggtaattttttccttcagtcttgaacatgtttttttaagctagtgaagctggtaaaagtaacatcaacagcatctctgaatggggaagaataaaaggcaatcctattttaaaaggtataaattttgcagggaatgtttgtataccgaaaattattccttatttacctgaaattcagattgaacctggcatttgcattttatctgacaactctgcctgtaaataaatgcagttcccttagtgaatcttcagttaaggaaatttccttgggctttctttaaaaatcaaatgtgaatacggtgtattaatcagctagggctgctgtaacaagtagcacagtcatgtggtttgagcagcaggaatgcctcctatcactgttctgagggctggaagtccaggcccgaggtgctggcagggctggcctggtgagggctctctctctgactggcctccatgtggtcttctctccgtggtcttccctctgtggtgttccctctgtgttcttccctccgtggtcttccctcagtttgctccttagatgtctccatcttcctgcatggacatcaattatactggatcagggccccactcttatgatgacctcatttatccttaataaacttcttaaaggctttatctccaaatacagtcacattcagagttaagccttcatccatgaattttgaatccataacaagcaggatagaattatttctgtcagtgtggcttggatttgaattgcttgcattgaaaacaaagcgtatcaataaaaggctcagcatggagctgaaactgcccctgctctgtcctcaggtctattgacggcaccaccaacgccatcacccttctttctgcaatcatctaaggtcccagctgtcttcccagtgaaaaacggctttacaataggaatgcttgtgatgtctagtaaagaaaagggatactagtgaaagaggtttgggggattctcacctgacctcgatacaagtttccgttttgaactggagtgaaatttctcccagtgtgtcaaatactggctgaagacaaagtgagaatttcccccaaatttatcttttgttttcatggccacagatattttatataaaatagttattttcaattggtgattctttttttactatttttatggggaccatccaagcatagttgaagtgaactaagaaaaaatccacgtcttttacgaagctatggcaatgcgtctaccaccttggccggcagggaggtttctcttgaaagaaacctctatcctattatttatcctagaataaataattctatcttattctttgcaccccctctgcatgcctgtgtgtgtttatacaccactgcacaagtcctcatacagtacatgattctaggacactgtctaatcatgtatactacatatgaaatacaacagttcagactttatgacaaatccttgcaataggttgtctttaacttgtatgtatgtggatttaatgtattttaagaactaaaatttttagctaagctaaaaagcctcagacgaattttcaggctttattatggatattgtagcacttctttttttttttttttttttttgaaacagagtcttgctctgttgcccaggctggagtgcaatggtgtgatctcggctcactgcaaccgccacctcccaggttcaagtgattctcctgcctcagcctccgagtagctgggactacaggcacgtggcaccacacccggccaattttttgtatttttagtagcatttagtggagagtagagtattttgtatttttgtgtttgccaggatgatcttgatctcttgacctcatgatctgcccgtctcagcctcccaaagtgctgggattacaggcatgagccactgtgcccagcctattgtagcacttctaatgcagagttagactttgcaaagtgtagagtgggtggcacatgacggaatcatacttgaaacacacagcagtatttggcaaactgtttggaagacactataaaaaaagacagaatgatacacggaaaataggagacaggcccgctatccttatttatgattaaatcctgaccacaaagcattctctctaacctcgcatttttatagctcataactctttgaagttaacttgggaaataaattttccaagattagtctcaactaaaaggcattttttctcttttgtttggaaacttggaaataagtttgtttttctgttttggtatgagtttgtatcagaaaaaaggatactttaattgtataagtcaatatttaaaacaaaaaatgagaaaattttcttaaaaatacttctttgggaagagagattttattggctcaataatttttttaaatgttaagcattttcaatcattatgttttctcatgaatgctaaaatgctttatctaaagccattagttcaaatttttgaaatagcaatatgttacaaccaaaatgtatcagaagttaaattatgggtatatacaaaattttaggttgaagcagatgtttgttgataaattacttaagatatttctctttgtttaatgctttacctttgtgtttaaaatactgccttttatttgtgcgtgtgtgtgtgtgtgtctttctataatttgtaaagccacgttgaatatctagtatatgctcaataaaatattgaacacctctgagtttccaatggtatcttgtaacatttttagtcgttttcagaggtggcaaagcaaatatttggagtcttgagcaaataaaagga

(19) United States
(12) Patent Application Publication
(10) Pub. No.: US 2025/0009906 A1
(43) Pub. Date: Jan. 9, 2025

(54) NOCICEPTOR-SPECIFIC GENE REGULATORY ELEMENTS FOR THE TREATMENT OF PAIN

(71) Applicants: President and Fellows of Harvard College, Cambridge, MA (US); The Brigham and Women's Hospital, Inc., Boston, MA (US)

(72) Inventors: William Renthal, Boston, MA (US); Sinisa Hrvatin, Cambridge, MA (US); Mark Aurel Nagy, Cambridge, MA (US); Eric C. Griffith, Cambridge, MA (US); Michael E. Greenberg, Cambridge, MA (US)

(73) Assignees: President and Fellows of Harvard College, Cambridge, MA (US); The Brigham and Women's Hospital, Inc., Boston, MA (US)

(21) Appl. No.: 18/711,241

(22) PCT Filed: Nov. 18, 2022
(86) PCT No.: PCT/US2022/080110

§ 371 Date: May 17, 2024

Related U.S. Application Data
(60) Provisional application No. 63/281,597, filed on Nov. 19, 2021.

(51) Int. Cl. A61K 48/00; C12N 15/86

(52) U.S. Cl. CPC A61K 48/0058; A61K 48/0075; C12N 15/86; C12N 2830/008; C12N 2830/48

(57) ABSTRACT

Aspects of the disclosure provide nucleic acids and compositions comprising gene regulatory elements (GREs) for specific expression in nociceptor cells. Other aspects of the disclosure relate to the use of vectors and compositions comprising the gene regulatory elements for treating or managing pain and other neurological diseases in a subject in need thereof.

### RELATED APPLICATIONS

This application claims priority under 35 U.S.C. § 119(e) to U.S. Provisional Application, U.S. Ser. No. 63/281,597, filed Nov. 19, 2021, the entire contents of which are incorporated herein by reference.

### FEDERALLY SPONSORED RESEARCH

This invention was made with Government support under DA048787 awarded by the National Institutes of Health. The Government has certain rights in the invention.

### REFERENCE TO AN ELECTRONIC SEQUENCE LISTING

The contents of the electronic sequence listing (H082470392WO00-SEQ-LJG.xml; Size: 61,225 bytes; and Date of Creation: Nov. 16, 2022) is herein incorporated by reference in its entirety.

### BACKGROUND

Chronic pain is a highly debilitating condition affecting over 20% of adults in the United States. It has been linked to numerous physical and mental conditions and contributes to high health care costs and lost productivity. It is often mediated by the abnormal activity of peripheral nociceptors whose cell bodies are housed in the dorsal root ganglion (DRG). Nociceptors are typically divided into three categories: small diameter peptidergic C-fibers, small diameter non-peptidergic C-fibers, and large diameter A-delta fibers. In contrast, A-beta peripheral sensory neurons mediate light touch, position sense, and pressure.

A current standard of care for the treatment of chronic pain is to use non-specific anesthetics, such as lidocaine, to silence nociceptor activity, but this approach is short-lived and leads to the inhibition of all peripheral sensory neurons in the affected area. Nociceptors are appealing candidates for targeted gene therapy-based interventions that aim to inhibit pain. A key feature is that the gene therapy vector (for example, an adeno-associated virus, AAV) should selectively express its genetic cargo (e.g., a transgene) within nociceptors so that other sensory functions are not diminished. Thus, nociceptor-restricted expression of the transgene of the gene therapy vector could enable the treatment and/or management of pain and peripheral neuropathies, such as diabetic neuropathy or chemotherapy-induced neuropathy, with increased safety, specificity, and duration.

### SUMMARY

The present disclosure describes engineered and naturally occurring gene regulatory elements (GRE) that are capable of driving expression of a heterologous gene (i.e., transgene) in desired cell types (e.g., peptidergic and non-peptidergic nociceptors in the DRG) with minimal expression in other DRG cell types. Using these GREs, novel gene therapies are enabled for next-generation therapeutics for treating and managing pain and peripheral neuropathies. Exemplary GREs described herein are listed as SEQ ID NOs: 1-5 in Table 1 and SEQ ID NOs: 17-22. These GREs may comprise binding sites for certain nociceptor-specific transcription factors.

The present disclosure herein, at least in part, relates to nucleic acids comprising an expression cassette, wherein the expression cassette comprises a GRE sequence as described herein, and a heterologous gene encoding a protein of interest. The protein of interest can be any protein. In certain embodiments, the protein of interest is a protein that provides a therapeutic benefit for the treatment or management of pain or a neurological disease. In some embodiments, the protein of interest is a pro-neurogenic peptide or a pro-neurogenic protein. In some embodiments, the protein of interest is an analgesic peptide, such as an endogenous opioid, a cannabinoid, or orexin. In some embodiments, the protein is a membrane protein. The membrane protein may be an ion channel, a chemogenetic channel, an optogenetic channel, or a custom-designed chemogenetic/optogenetic channel. In some embodiments, the ion channel is an inhibitory ion channel, for example, a Kir2.1 inward-rectifier potassium ion channel. In some embodiments, the chemogenetic channel is an inhibitory chemogenetic channel, including Cation-Permeable Glycine Receptors, Ivermectin-activated Cation-Permeable Glycine Receptors, Pharmacologically Selective Actuator Modules (PSAMs), hM4Di, and PSAM-GlyR. Table 2 lists sequences of exemplary heterologous genes and proteins of interest that may be used in gene therapy vectors for the treatment or management of pain. In some embodiments, the chemogenetic channel has a sequence that is at least 70% identical to any one of SEQ ID NOs: 7, 9 and 11, and is encoded by a nucleotide sequence having at least 70% identity with any one of SEQ ID NOs: 6, 8 and 10.

The presence of the GRE enables the selective expression of the protein of interest in peptidergic and non-peptidergic nociceptors compared to other cells of the dorsal root ganglion (DRG) or compared to other cells of the central nervous system (CNS). In some embodiments, at least 70% of the DRG cells where the protein of interest is expressed are peptidergic and non-peptidergic nociceptors. In some embodiments, expression of the protein of interest is at least 2 times, at least 5 times, or at least 10 times higher in peptidergic and non-peptidergic nociceptors than in other cells of the DRG or CNS. In some embodiments, the GRE sequence has at least 80% identity to any one of SEQ ID NOs: 1-5, or a fragment thereof. In some embodiments, the GRE comprises any one of SEQ ID NOs: 1-5, or a fragment thereof. In some embodiments, the GRE consists of any one of SEQ ID NOs: 1-5, or a fragment thereof. In some embodiments, the GRE sequence is between 25 and 1,000 nucleotides. In some embodiments, the GRE comprises binding sites for one or more transcription factors. Examples of transcription factors that can be used in the present disclosure include, but are not limited to, Pit1 (POU Class 1 factors), Oct1 (POU Class 2 factors), HoxA11, GR, Arnt1, Srebf2, PU.1, and combinations thereof.

In some embodiments, the expression cassette further comprises at least a promoter operably linked to a nucleotide sequence encoding a 5′ untranslated region (5′ UTR), an intron, an enhancer, a post-transcriptional regulatory element, a 3′ UTR and/or a polyA signal. The promoter may be a beta globin promoter (pBG), CAG promoter (pCAG), AVIL, OPRM1, TAC1, SCN9A, SCN10A, a CMV promoter (e.g., minimal CMV promoter), a human synapsin promoter, a chicken beta actin, a PGK promoter, an Ef1a promoter, a ubiquitin promoter, a TATA-box containing promoter, a Calca promoter, a Taca promoter, a Trpv1 promoter, and variants thereof. In some embodiments, the post-transcriptional regulatory element is a Woodchuck Hepatitis Virus Posttranscriptional Regulatory Element (WPRE).

In some embodiments, the GRE is located in the expression cassette, upstream of the promoter and heterologous gene encoding the protein of interest. In some embodiments, the expression cassette is flanked by two adeno-associated virus (AAV) inverted terminal repeats (ITRs), wherein the AAV ITRs are from a serotype selected from the group consisting of AAV1, AAV2, AAV3, AAV4, AAV5, AAV6, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, and AAV-PHP.eB.

In some aspects, the present disclosure provides a vector comprising a nucleic acid described herein (for example, an expression cassette as described above). In some embodiments, the vector is a viral vector. In some embodiments, the viral vector is an AAV vector, a herpes vector, or a lentiviral vector. In some embodiments, the vector is a non-viral vector, such as a plasmid, phage, transposon, cosmid, chromosome, or artificial chromosome. In some embodiments, the non-viral vector is a plasmid.

In some aspects, the present disclosure provides a recombinant adeno-associated viral (rAAV) vector comprising a gene regulatory element (GRE) sequence, a nucleic acid, or an expression cassette, as described herein.

In some aspects, the disclosure provides a pharmaceutical composition comprising a nucleic acid described herein, an expression cassette described herein, a vector described herein, or a rAAV described herein. In some embodiments, the isolated nucleic acid, expression cassette, vector, or rAAV described herein is part of a delivery vehicle, such as a lipid microparticle, a lipid nanoparticle, a polymeric microparticle, a polymeric nanoparticle, a liposome, an endosome, a micelle, a vesicle, a gold nanoparticle, a carbon nanotube, a quantum dot, a magnetic nanoparticle, a dendrimer, a calcium phosphate vehicle, a DNA-coated microparticle or a polymer. In some embodiments, the pharmaceutical composition further comprises a pharmaceutically acceptable carrier.

In some aspects, the present disclosure also provides a method for selectively expressing a protein of interest in a peptidergic and/or non-peptidergic nociceptor, the method comprising contacting the nociceptor with a nucleic acid described herein, an expression cassette described herein, a vector described herein, an rAAV described herein, or a pharmaceutical composition described herein.

In some embodiments, the present disclosure provides nucleic acids, expression cassettes, vectors, rAAVs, and compositions for use in the treatment and/or management of a neurological disease or condition (e.g., pain) in a subject.

In some embodiments, the present disclosure provides methods of treating or managing a neurological disease or condition (e.g., pain) in a subject, the method comprising administering to the subject a nucleic acid described herein, an expression cassette described herein, a vector described herein, an rAAV described herein, or a pharmaceutical composition described herein. In some embodiments, the administering results in a reduction of abnormal activity of nociceptors in the subject. In some embodiments, the subject is a non-human mammal. In some embodiments, the subject is a human. In some embodiments, the step of administering to a subject is through injection. In some embodiments, the injection comprises an intracerebroventricular (ICV) injection, an intrathecal injection, an intravenous (IV) injection, or an injection into the cisterna magna (ICM). In some embodiments, the neurological disease or condition being treated is pain, back pain, chronic pain, a peripheral neuropathy, a diabetic neuropathy, neuralgia, a chemotherapy-induced neuropathy, migraine, or trigeminal neuralgia.

The details of one or more embodiments of the present disclosure are set forth in the description below. Other features or advantages of the present disclosure will be apparent from the following drawing and detailed description of certain embodiments and also from the appended claims.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 shows the expression of mPEP1_3-mCMV-GFP in the mouse DRG.

FIG. 2 shows the expression of mNP9_3-mCMV-GFP in the mouse DRG.

FIG. 3 shows GFP expression in mouse brain and DRG in the presence of the PEP1_17 GRE (SEQ ID NO. 5).

FIG. 4 is a graph showing the percentage of EGFP positive nociceptors (Tac1+ cells) in the total EGFP+ cells in the DRG in mice that received AAV-GFP vector comprising PEP1-17 or PEP1-3 GRE.

The accompanying drawings, which are incorporated in and constitute a part of this specification, illustrate certain embodiments, and together with the written description, serve to provide non-limiting examples of certain aspects of the compositions and methods disclosed herein.

### DETAILED DESCRIPTION

### Definitions

Gene regulatory elements (GREs), or DNA regulatory elements, as used herein, refer to a variety of DNA sequences that are involved in the regulation of gene expression. For example, a GRE may rely on the interactions involving DNA, cellular proteins (e.g., histones), and/or transcription factors to regulate gene expression. A GRE can be a cis-regulatory element. Cis-regulatory elements are regions of non-coding DNA which regulate the transcription of nearby genes. Cis-regulatory elements are found in the vicinity of the genes that they regulate. Cis-regulatory elements typically regulate gene transcription by binding to transcription factors.

Promoters and enhancers are the primary genomic regulatory components of gene expression. Promoters are DNA regions typically within 1-2 kilobases (kb) of a gene's transcription start site (TSS); they contain short regulatory elements (DNA motifs) necessary to assemble RNA polymerase transcriptional machinery. An enhancer, as used herein, refers to DNA sequences, which are located more distal to the transcription start site as compared to a promoter, capable of interacting with site-specific transcription factors to regulate gene expression in a cell-type specific manner. Enhancers confer cell-specific gene expression regulation by binding to the collection of transcription factors in a cell, which leads to transcriptional activation or inhibition through various mechanisms, e.g., recruitment of epigenetic enzymes that catalyze post-translational histone modifications, and recruitment of cofactors that promote DNA looping. Enhancers can be identified in the vicinity of the gene they regulate, or at a distance of hundreds of kilobases from their target genes. Multiple enhancers can act additively and redundantly to regulate gene expression (e.g., Doane et al., Regulatory elements in molecular networks, Wiley Interdiscip Rev Syst Biol Med. 2017 May; 9(3)). Enhancers can be identified in the transcriptionally active sequences of their target genes. A transcriptionally active sequence, as used herein, refers to a region of DNA in a chromosome in which the DNA is in open chromatin conformation such that the sequence is exposed, thereby allowing binding of transcription factors and transcription to take place.

An expression cassette, as used herein, refers to a component of a vector DNA comprising a protein coding sequence to be expressed by a cell having the vector and its regulatory sequences. Once delivered to the target cell, the expression cassette directs the cell's machinery to make RNA and/or protein(s).

A nucleic acid sequence, as used herein, refers to a DNA or RNA sequence. Proteins and nucleic acids of the disclosure can be isolated. As used herein, the term isolated means artificially produced. As used herein with respect to nucleic acids, the term isolated means: (i) amplified in vitro by, for example, the polymerase chain reaction (PCR); (ii) recombinantly produced by cloning; (iii) purified, for example, by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein, with respect to proteins or peptides, the term “isolated” refers to a protein or peptide that has been isolated from its natural environment or artificially produced (e.g., by chemical synthesis, by recombinant DNA technology, etc.).

As used herein, the term “sequence identity” refers to the percentage of nucleic acid (or amino acid) residues of a candidate sequence that are identical to the nucleic acid (or amino acid) residues of a reference sequence, after aligning the sequences and introducing gaps, if necessary, to achieve the maximum percent identity (e.g., gaps can be introduced in one or both of the candidate and reference sequences for optimal alignment and non-homologous sequences can be disregarded for comparison purposes). Alteration of the amino acid sequence or nucleic acid coding sequences can be obtained by deletion, addition, or substitution of residues of the reference sequence. Alignment for purposes of determining percent identity can be achieved in various ways that are within the skill of one in the art, for instance, using publicly available computer software, such as BLAST, BLAST-2, BLAST-P, BLAST-N, BLAST-X, WU-BLAST-2, ALIGN, ALIGN-2, CLUSTAL, or Megalign (DNASTAR) software. Those skilled in the art can determine appropriate parameters for measuring alignment, including any algorithms needed to achieve maximal alignment over the full length of the sequences being compared. For instance, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

100×(fraction of A/B)
where A is the number of amino acid (or nucleic acid) residues scored as identical in the alignment of the candidate sequence and the reference sequence, and where B is the total number of amino acid (or nucleic acid) residues in the reference sequence. In particular, a reference sequence aligned for comparison with a candidate sequence can show that the candidate sequence exhibits from, e.g., 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, 95%, 98%, 99%, or 100% of the length of the reference sequence. When a position in the candidate sequence is occupied by the same amino acid (or nucleic acid) residue as the corresponding position in the reference sequence, then the molecules are identical at that position.

As used herein, a substitution in a nucleic acid sequence occurs when one nucleotide or base pair, or one set of consecutive nucleotides or base pairs, within the nucleic acid sequence is replaced by another. As used herein, an insertion in a nucleic acid sequence occurs when one nucleotide or base pair, or one set of consecutive nucleotides or base pairs, is inserted (i.e. added) between two consecutive nucleotides or base pairs within the nucleic acid sequence, or at the 3′ or 5′ end of the nucleic acid sequence. As used herein, a deletion in a nucleic acid sequence occurs when one nucleotide or base pair, or one set of consecutive nucleotides or base pairs, is deleted (i.e., removed) from the nucleic acid sequence between two nucleotides or base pairs of the nucleic acid sequence, or from the 3′ or 5′ end of the nucleic acid sequence.

As used herein, post-transcriptional regulatory element refers to a nucleic acid sequence that, when transcribed, adopts a tertiary structure that enhances expression of a gene.

As used herein with respect to AAVs, the term “isolated” refers to an AAV that has been artificially produced, engineered, or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, a rAAV having a capsid appropriate for the tissue being targeted can be selected.

A single stranded AAV (ssAAV), as used herein, refers to a rAAV with the coding sequence and complementary sequence of the heterologous gene (i.e., transgene) expression cassette on separate strands and packaged in separate viral capsids. A scAAV, as used herein, refers to a rAAV with both the coding and complementary sequence of the heterologous gene (i.e., transgene) expression cassette are present on the single strand of an AAV genome. The coding region of a scAAV was designed to form an intra-molecular double-stranded DNA template. Upon infection, rather than waiting for cell mediated synthesis of the second strand, the two complementary halves of scAAV will associate to form one double-stranded DNA (dsDNA) unit that is ready for immediate replication and transcription.

A “host cell” refers to any cell that harbors, or is capable of harboring, a substance of interest. Often a host cell is a mammalian cell. A host cell may be used as a recipient of an AAV helper construct, an AAV plasmid, an accessory function vector, or other transfer DNA associated with the production of recombinant AAVs. The term includes the progeny of the original cell which has been transfected. Thus, a “host cell,” as used herein, may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation or engineering.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some vectors, the nucleic acid segment to be transcribed is positioned under the transcriptional control of a promoter. The term “expression vector or construct” means any type of genetic construct containing a nucleic acid in which part or all of the nucleic acid encoding sequence is capable of being transcribed.

As used herein, “carrier” includes any and all solvents, dispersion media, vehicles, solvents, coatings, diluents, antibacterial and antifungal agents, isotonic and absorption delaying agents, buffers, carrier solutions, suspensions, colloids, and the like. The use of such media and agents for pharmaceutically active substances is well known in the art. Supplemental active ingredients can also be incorporated into the compositions. The phrase “pharmaceutically acceptable” refers to molecular entities and compositions that do not produce an allergic or similar untoward reaction when administered to a host.

As used herein, the term “pain” refers to an unpleasant sensory and emotional experience associated with actual or potential tissue damage, or described in terms of such damage. Pain can be classified to include transient, acute and chronic pain. Acute and chronic pain are further categorized based on organ or tissue localization, whether it is malignant, e.g., having a cancerous origin, or nonmalignant. Furthermore, pain may be characterized as nociceptive, neuropathic or a combination thereof. Non-limiting examples of pain that are contemplated by the present disclosure include posttherapeutic neuralgia, posttherapeutic neuralgia, diabetic neuropathy, postmastectomy pain syndrome, stump pain, reflex sympathetic dystrophy, trigeminal neuralgia, neuropathic pain, orofacial neuropathic pain, diabetic neuropathy, causalgia, phantom limb pain, osteoarthritis, rheumatoid arthritis, pain associated with cancer, pain associated with HIV, fibromyalgia syndrome, tension myalgia, Guillian-Barre syndrome, Meralgia paraesthetica, burning mouth syndrome, fibrocitis, myofascial pain syndrome, idiopathic pain disorder, temporomandibular joint syndrome, atypical odontalgia, loin pain, haematuria syndrome, non-cardiac chest pain, low back pain, chronic nonspecific pain, psychogenic pain, musculoskeletal pain disorder, chronic pelvic pain, nonorganic chronic headache, tension-type headache, cluster headache, migraine and other conditions associated with chronic cephalic pain, complex regional pain syndrome, vaginismus, nerve trunk pain, somatoform pain disorder, cyclical mastalgia, chronic fatigue syndrome, multiple somatization syndrome, chronic pain disorder, somatization disorder, tabes dorsalis, spinal cord injury, central pain, posttherapeutic pain, noncardiac chest pain, irritable bowel syndrome, central post-stroke pain, Syndrome X, facial pain, idiopathic pain disorder, posttraumatic rheumatic pain modulation disorder (fibrositis syndrome), hyperalgesia, inflammatory pain and Tangier disease.

As used herein, a neurological disease, condition, or disorder refers to any disease, condition, or disorder of the nervous system and/or visual system. Neurological diseases, conditions, or disorders include diseases, conditions, or disorders that involve the central nervous system (brain, brainstem and cerebellum), the peripheral nervous system (including cranial nerves), and the autonomic nervous system (parts of which are located in both central and peripheral nervous system). A neurological disease, condition, or disorder includes but is not limited to: acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury, brain abscess; brain injury; brain tumors (including glioblastoma multiforme) spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; DandyWalker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid colt leukodystrophy; Guillain-Barre Syndrome; HTLV-1-associated myelopathy; Hallervorden-Spatz disease; head injury; headache; hemifacial spasm; hereditary spastic paraplegia; heredopathia atactic a polyneuritiformis; herpes zoster oticus; herpes zoster; Hirayama syndrome; HIV associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myosins; incontinentia pigmenti; infantile phytanic acid storage disease; infantile refsum disease; infantile spasms; inflammatory myopatby; intracranial cyst; intracranial hypertension; Joubert syndrome; Keams-Sayre syndrome; Kennedy disease Kinsboume syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuronlyotonia; neuronal ceroid lipofuscinosis; neuronal migration disorders; Niemann-Piet disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; a neurodegenerative disease or disorder (Parkinson's disease, Huntington's disease, Alzheimer's disease, amyotrophic lateral sclerosis (ALS), dementia, multiple sclerosis and other diseases and disorders associated with neuronal cell death); paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; phone sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocalleukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal attains; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infaret dementia); vasculitis including temporal arteritis; Von Hippel-Lindau disease; Wallenberg's syndrome; Werdnig-Hoffman disease; West syndrome; whiplash; Williams syndrome: Wildon's disease; and Zellweger syndrome. Additional neurological diseases, disorders and conditions also contemplated by the present disclosure include ischemic disease, diabetic neuropathy, anti-cancer-agent-intoxicated neuropathy, retinal pigment degeneration, glaucoma, an anoxic episode, an injury to the brain and other parts of the CNS caused by trauma or other injury, a blow to the head, a spinal injury, a thromboembolic or hemorrhagic stroke, a cerebral vasospasm, hypoglycemia, cardiac arrest, cerebral ischemia or cerebral infarction, ischemic, hypoxic or anoxic brain damage, spinal cord injury, tissue ischemia and reperfusion injury.

As used herein, the term “treating” or “treatment” refers to the application or administration of a composition including one or more active agents (e.g., nucleic acids, vectors, rAAVs or compositions described herein) to a subject, for example a subject who is diagnosed with disease or condition (e.g., pain, back pain, chronic pain, a peripheral neuropathy, a diabetic neuropathy, neuralgia, a chemotherapy-induced neuropathy, migraine or trigeminal neuralgia), with the purpose to cure, heal, alleviate, relieve, alter, remedy, ameliorate, improve, or affect the disease or condition, or a symptom of the disease or condition. Alleviating a target disease or condition includes delaying or preventing the development or progression of the disease or condition, or reducing the severity of the disease or condition.

As used herein, a protein of interest is a protein or peptide that presents an interest in the treatment, management, or diagnosis of a medical disease, disorder, or condition, or in an experimental assay. Any gene that one of skill in art would like to express in peptidergic and non-peptidergic nociceptors in the DRG can be delivered using the isolated nucleic acids described herein.

The present disclosure describes isolated gene regulatory elements (GRE) specific to peptidergic and non-peptidergic nociceptors. When combined to a heterologous gene(s) in an expression cassette, the GREs of the present disclosure can direct the selective expression of the heterologous gene(s) in peptidergic and non-peptidergic nociceptors, while restricting expression of the heterologous gene(s) in other DRG cell types. This specific targeting of nociceptors is relevant for next-generation gene therapies that aim to treat and/or manage pain and other neurological diseases that result from an abnormal activity of nociceptors. Side effects of such therapies on other sensory functions (e.g., light touch, temperature, pressure, and position sensing) which are mediated by other neurological cells (i.e., cells that are not nociceptors) are thus reduced by limiting exposure of these other neurological cells to the treatment.

Some aspects of the disclosure provide isolated nucleic acids, expression cassettes, vectors, cells, or compositions comprising one or more of the GREs described herein. In some aspects, the GREs within the nucleic acids, vectors, cells or compositions of the present disclosure are comprised within an expression cassette, in addition to a promoter(s), a heterologous gene(s) encoding a protein(s) of interest, a post-transcriptional regulatory element(s), and/or a polyA signal. In some embodiments, the expression cassette is flanked by two adeno-associated virus (AAV) inverted terminal repeats (ITRs).

In some embodiments, the present disclosure provides nucleic acids, expression cassettes, vectors, rAAVs and compositions for use in the treatment and/or management of a neurological disease or condition (e.g., pain) in a subject.

Other aspects of the present disclosure provide methods of treatment and/or management of pain and other neurological diseases in a subject, through administration to the subject of compositions comprising an expression cassette, nucleic acid, or vector comprising one or more of the GRE sequences described herein, and a therapeutic heterologous gene.

### I. Genomic Regulatory Elements

The present disclosure provides an isolated nucleic acid comprising one or more gene regulatory element (GRE) sequences. Table 1 lists exemplary GRE sequences (SEQ ID NOs: 1-5) and SEQ ID NOs: 17-22. In some embodiments, the isolated nucleic acid comprises a gene regulatory element sequence that has at least 80% identity to SEQ ID NOs: 1-5, or a fragment thereof. In some embodiments, an isolated nucleic acid comprises a gene regulatory element sequence that has at least 85% identity to any one of SEQ ID NOs: 1-5 and SEQ ID NOs: 17-22, or a fragment thereof. In some embodiments, the gene regulatory element sequence has at least 90%, 95%, 98%, or 99% identity to any one of SEQ ID NOs: 1-5 and SEQ ID NOs: 17-22, or a fragment thereof. In some embodiments, the GRE sequence has at least 80%, at least 85%, at least 90%, at least 95%, at least 98%, at east 99%, or 100% identity to the sequence corresponding to chr3:147,092,200-147,092,400 (SEQ ID NO: 17) in mouse (genome version mm10), or a fragment thereof. In some embodiments, the GRE sequence has at least 80%, at least 85%, at least 90%, at least 95%, at least 98%, at east 99%, or 100% identity to the sequence corresponding to chr1:83770144-83770344 (SEQ ID NO: 18) in human (genome version hg38), or a fragment thereof. In some embodiments, the GRE sequence has at least 80% identity, at least 85%, at least 90%, at least 95%, at least 98%, at east 99%, or 100% to the sequence corresponding to chr9:32,510,922-32,511,149 (SEQ ID NO: 19) in mouse (genome version mm10), or a fragment thereof. In some embodiments, the GRE sequence has at least 80%, at least 85%, at least 90%, at least 95%, at least 98%, at east 99%, or 100% identity to the sequence corresponding to chr11:128724578-128724853 (SEQ ID NO: 20) in human (genome version hg38), or a fragment thereof. In some embodiments, the GRE sequence has at least 80% identity, at least 85%, at least 90%, at least 95%, at least 98%, at east 99%, or 100% to the sequence corresponding to chr17:14,364,518-14,364,696 (SEQ ID NO: 21) in mouse (genome version mm10), or a fragment thereof. In some embodiments, the GRE sequence has at least 80%, at least 85%, at least 90%, at least 95%, at least 98%, at east 99%, or 100% identity to the sequence corresponding to chr6:168541483-168567667 (SEQ ID NO: 22) in human (genome version hg38), or a fragment thereof. In some embodiments, the GRE sequence has at least 85% identity to any one of SEQ ID NOs: 17-22, or a fragment thereof. In some embodiments, the GRE sequence has at least 90%, 95%, 98%, or 99% identity to any one of SEQ ID NOs: 17-22, or a fragment thereof. In some embodiments, the gene regulatory element sequence comprises any one of SEQ ID NOs: 1-5, or a fragment thereof, or any one of SEQ ID NOs: 17-22, or a fragment thereof. In some embodiments, the GRE consists of any one of SEQ ID NOs: 1-5, or a fragment thereof, or any one of SEQ ID NOs: 17-22, or a fragment thereof. In some embodiments, the gene regulatory element sequence comprises at least one modification relative to any one of SEQ ID NOs: 1-5, or SEQ ID NOs 17-22. In some embodiments, the gene regulatory element sequence comprises at least two, at least three, at least four, or at least five modifications relative to any one of SEQ ID NOs: 1-5, or SEQ ID NOs 17-22. Modifications in a genomic sequence include one or more of an insertion, a deletion, and/or a substitution. In some embodiments, the GRE comprises a sequence that is at least 1,000 nucleotides, at least 950 nucleotides, at least 900 nucleotides, at least 850 nucleotides, at least 800 nucleotides, at least 750 nucleotides, at least 700 nucleotides, at least 650 nucleotides, at least 600 nucleotides, at least 550 nucleotides, at least 500 nucleotides at least 450 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, at least 25 nucleotides, or at least 20 nucleotides, of any one of SEQ ID NOs: 1-5, or SEQ ID NOs: 17-22.

Gene regulatory elements may rely on interactions involving DNA and cellular proteins (e.g., histones, transcription factors) to regulate gene expression. The GREs described herein were identified by mapping chromatin accessibility across the genome in nociceptors and other cell types, and selecting regions of chromatin that were significantly more accessible in nociceptors than other DRG cell types. The GREs of the present disclosure comprise one or more binding sites for certain transcription factors that enable nociceptor-specific expression. For example, mouse PEP1_3, or mPEP1_3, (SEQ ID NO. 1) contains binding sites for the following transcription factors: Pit1 (POU Class 1 factor), Oct1 (POU Class 2 factor), HoxA11, and glucocorticoid receptor (GR). Mouse NP9_3 (mNP9_3, SEQ ID NO. 3) contains binding sites for the following transcription factors: Aryl hydrocarbon receptor nuclear translocator-like protein 1 (ARNTL), Sterol Regulatory Element Binding Transcription Factor 2 (SREBF2), and PU.1.

Transcription factors (TFs) work alone or with other proteins in a complex, by promoting (as an activator), or blocking (as a repressor) the recruitment of RNA polymerase (the enzyme that performs the transcription of genetic information from DNA to RNA) to specific genes. A defining feature of TFs is that they contain at least one DNA-binding domain (DBD), which attaches to a specific sequence of DNA adjacent to the genes that they regulate. Transcription factors bind to either enhancer or promoter regions of DNA adjacent to the genes that they regulate. Depending on the transcription factor, the transcription of the adjacent gene is either up- or down-regulated. Transcription factors use a variety of mechanisms for the regulation of gene expression. These mechanisms include: (i) stabilization or blocking of RNA polymerase binding to DNA, (ii) catalysis of the acetylation or deacetylation of histone proteins, and/or (iii) recruitment of coactivator or corepressor proteins to the transcription factor DNA complex.

In some embodiments, a GRE sequence comprises one or more transcription factor binding sites. In some embodiments, the isolated nucleic acid comprises one or more GREs which are cis-regulatory elements. Cis-regulatory elements are regions of non-coding DNA which regulate the transcription of neighboring genes. Cis-regulatory elements are found in the vicinity of the genes that they regulate. Cis-regulatory elements typically regulate gene transcription by binding to transcription factors. In some embodiments, the GREs impart cell-specific gene expression capabilities. In some embodiments, the GREs comprise one or more (e.g., 1, 2, 3, 4, 5, 6, 7, 9, or more) enhancers. In some embodiments, the isolated nucleic acid comprises more than one enhancer, and the more than one enhancer are the same enhancers or different enhancers. In some embodiments, the one or more enhancers comprise a sequence that is at least 70% identical to any one of SEQ ID NOs: 1-5, or SEQ ID NOs: 17-22. In some embodiments, the one or more enhancers comprise a sequence that is at least 75% identical to any one of SEQ ID NOs: 1-5, or SEQ ID NOs: 17-22. In some embodiments, the one or more enhancers comprise a sequence that is at least 80% identical to any one of SEQ ID NOs: 1-5, or SEQ ID NOs: 17-22. In some embodiments, the one or more enhancers comprise a sequence that is at least 90% identical to any one of SEQ ID NOs: 1-5, or SEQ ID NOs: 17-22. In some embodiments, the one or more enhancers comprise a sequence that is at least 95% identical to any one of SEQ ID NOs: 1-5, or SEQ ID NOs: 17-22. In some embodiments, the one or more enhancers comprise a sequence that is at least 98% identical to any one of SEQ ID NOs: 1-5, or SEQ ID NOs: 17-22. In some embodiments, the one or more enhancers comprise a sequence that is at least 99% identical to any one of SEQ ID NOs: 1-5, or SEQ ID NOs: 17-22. In some embodiments, the one or more enhancers comprise a sequence that is identical to any one of SEQ ID NOs: 1-5, or SEQ ID NOs: 17-22. In some embodiments, the one or more enhancers comprise binding sites for one or more transcription factors. Examples of transcription factors that can be used in the present disclosure include, but are not limited to, Pit1 (POU Class 1 factors), Oct1 (POU Class 2 factors), HoxA11, GR, Arnt1, Srebf2, PU.1, Rbfox3, RUNX1, ISL1, ISL2, HMX1, HOXD1, and combinations thereof.

In some embodiments, the one or more GREs comprise one or more enhancers that enable selective expression of the protein in peptidergic and non-peptidergic nociceptors compared to other cells of the dorsal root ganglion (DRG) or compared to other cells of the central nervous system (CNS). In some embodiments, expression of the protein of interest is at least 1.5 times, at least 2 times, at least 3 times, at least 5 times, at least 8 times, at least 10 times, at least 15 times, at least 20 times, at least 25 times, at least 50 times, at least 75 times, or at least 100 times higher in peptidergic and non-peptidergic nociceptors than in other cells of the DRG or CNS on average.

In some embodiments, at least 50% of the DRG cells where the protein of interest is expressed are peptidergic and non-peptidergic nociceptors. In some embodiments, at least 60% of the DRG cells where the protein of interest is expressed are peptidergic and non-peptidergic nociceptors. In some embodiments, at least 70% of the DRG cells where the protein of interest is expressed are peptidergic and non-peptidergic nociceptors. In some embodiments, at least 75% of the DRG cells where the protein is expressed are peptidergic and non-peptidergic nociceptors. In some embodiments, at least 80% of the DRG cells where the protein of interest is expressed are peptidergic and non-peptidergic nociceptors. In some embodiments, at least 85% of the DRG cells where the protein is expressed are peptidergic and non-peptidergic nociceptors. In some embodiments, at least 90% of the DRG cells where the protein of interest is expressed are peptidergic and non-peptidergic nociceptors. In some embodiments, at least 95% of the DRG cells where the protein of interest is expressed are peptidergic and non-peptidergic nociceptors. In some embodiments, at least 98% of the DRG cells where the protein of interest is expressed are peptidergic and non-peptidergic nociceptors. In some embodiments, at least 99% of the DRG cells where the protein of interest is expressed are peptidergic and non-peptidergic nociceptors.

### II. Isolated Nucleic Acids

Some aspects of the present disclosure provide an isolated nucleic acid comprising one or more of the GREs described herein. In some embodiments, the isolated nucleic acid comprises an expression cassette comprising the GRE. In some embodiments, the expression cassette further comprises a heterologous gene(s) encoding a protein(s) of interest (e.g., a pro-neurogenic protein, or an ion channel), a promoter(s), a 5′ UTR, an intron(s), an enhancer(s), a post-transcriptional regulatory element(s), and/or a polyA signal, a post-transcriptional regulatory element, a 3′ UTR and/or a polyA signal. In some embodiments, the expression cassette comprises a promoter(s). In some embodiments, the expression cassette comprises a heterologous gene(s) encoding a protein(s) of interest. In some embodiments, the expression cassette comprises a post-transcriptional regulatory element(s). In some embodiments, the expression cassette comprises a polyA signal. In some embodiments, the expression cassette is flanked by two adeno-associated virus (AAV) inverted terminal repeats (ITRs).

In some embodiments, the polyA signal has a sequence that has at least 80% identity to SEQ ID NO: 14. In some embodiments, the polyA signal has a sequence that has at least 85% identity to SEQ ID NO: 14. In some embodiments, the polyA signal has a sequence that has at least 90%, 95%, 98%, or 99% identity to SEQ ID NO: 14. In some embodiments, the polyA signal has a sequence that comprises SEQ ID NO: 14. In some embodiments, the polyA signal has a sequence that consists of SEQ ID NO: 14. In some embodiments, an ITR has a sequence that has at least 80% identity to one of SEQ ID NO: 15 or SEQ ID NO: 16. In some embodiments, an ITR has a sequence that has at least 85% identity to one of SEQ ID NO: 15 or SEQ ID NO: 16. In some embodiments, an ITR has a sequence that has at least 90%, 95%, 98%, or 99% identity to one of SEQ ID NO: 15 or SEQ ID NO: 16. In some embodiments, an ITR has a sequence that comprises one of SEQ ID NO: 15 or SEQ ID NO: 16. In some embodiments, an ITR has a sequence that consists of one of SEQ ID NO: 15 or SEQ ID NO: 16.

SEQ ID NO: 14 (polyA signal):
AACTTGTTTATTGCAGCTTATAATGGTTACAAATAAAGCAATAGC
ATCACAAATTTCACAAATAAAGCATTTTTTTCACTGC
SEQ ID NO: 15 (5′ ITR):
CCTGCAGGCAGCTGCGCGCTCGCTCGCTCACTGAGGCCGCCCGGG
CAAAGCCCGGGCGTCGGGCGACCTTTGGTCGCCCGGCCTCAGTGA
GCGAGCGAGCGCGCAGAGAGGGAGTGGCCAACTCCATCACTAGGG
GTTCCT
SEQ ID NO: 16 (3′ ITR):
AGGAACCCCTAGTGATGGAGTTGGCCACTCCCTCTCTGCGCGCTC
GCTCGCTCACTGAGGCCGGGCGACCAAAGGTCGCCCGACGCCCGG
GCTTTGCCCGGGCGGCCTCAGTGAGCGAGCGAGCGCGCAGCTGCC
TGCAGG

In some embodiments, the promoter is operably linked to a nucleotide sequence encoding a 5′ untranslated region (5′ UTR). In some embodiments, the promoter is a tissue/cell-specific promoter. A tissue/cell specific promoter, as used herein, refers to a promoter that has activity in only certain cell types. In some embodiments, the promoter used in the nucleic acid described herein is specific to peptidergic and/or non-peptidergic nociceptors. Use of a tissue/cell-specific promoter in the nucleic acid described herein can restrict unwanted expression of the heterologous gene in cells that are not nociceptors, as well as facilitate persistent expression of the heterologous gene in peptidergic and/or non-peptidergic nociceptors. In some embodiments, the promoter is a beta globin promoter (pBG), CAG promoter (pCAG), AVIL, OPRM1, TAC1, SCN9A, SCN10A, a CMV promoter (e.g., minimal CMV promoter), a human synapsin promoter, a chicken beta actin, a PGK promoter, an Ef1a promoter, a ubiquitin promoter, a TATA-box containing promoter, a Calca promoter, a Taca promoter, a Trpv1 promoter, and variants thereof. In some embodiments, the promoter is a murine CMV promoter. In some embodiments, the promoter has a sequence that has at least 80% identity to SEQ ID NO. 12. In some embodiments, the promoter has a sequence that has at least 85% identity to SEQ ID NO. 12. In some embodiments, the promoter has a sequence that has at least 90%, 95%, 98%, or 99% identity to SEQ ID NO. 12. In some embodiments, the promoter has a sequence that comprises SEQ ID NO. 12. In some embodiments, the promoter has a sequence that consists of SEQ ID NO. 12.

SEQ ID NO. 12 (CMV promoter):
CGTTACATAACTTACGGTAAATGGCCCGCCTGGCTGACCGCCCAA
CGACCCCCGCCCATTGACGTCAATAATGACGTATGTTCCCATAGT
AACGCCAATAGGGACTTTCCATTGACGTCAATGGGTGGAGTATTT
ACGGTAAACTGCCCACTTGGCAGTACATCAAGTGTATCATATGCC
AAGTACGCCCCCTATTGACGTCAATGACGGTAAATGGCCCGCCTG
GCATTATGCCCAGTACATGACCTTATGGGACTTTCCTACTTGGCA
GTACATCTACGTATTAGTCATCGCTATTACCATGGTGATGCGGTT
TTGGCAGTACATCAATGGGCGTGGATAGCGGTTTGACTCACGGGG
ATTTCCAAGTCTCCACCCCATTGACGTCAATGGGAGTTTGTTTTG
GCACCAAAATCAACGGGACTTTCCAAAATGTCGTAACAACTCCGC
CCCATTGACGCAAATGGGCGGTAGGCGTGTACGGTGGGAGGTCTA
TATAAGCAGAGCT

In some embodiments, the promoter is an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other promoters have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natd. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)).

In some embodiments, the heterologous gene encodes a protein of interest. The protein of interest can be any protein. Preferably, the protein of interest is a protein that provides a therapeutic benefit for the treatment or management of pain or a neurological disease. Table 2 lists exemplary heterologous genes encoding proteins of interest for the treatment or management of pain, that may be used as transgenes in a gene therapy treatment targeting nociceptors. In some embodiments, the protein of interest is a pro-neurogenic peptide or a pro-neurogenic protein. In some embodiments, the protein of interest is an analgesic peptide. In some embodiments, the protein of interest is an endogenous opioid, a cannabinoid, or orexin. In some embodiments, the protein of interest is a membrane protein. In some embodiments, the membrane protein is an ion channel, a chemogenetic channel, an optogenetic channel or a custom-designed chemogenetic/optogenetic channel. In some embodiments, the ion channel is an inhibitory ion channel. In some embodiments, the inhibitory ion channel is a Kir2.1 inward-rectifier potassium ion channel. In some embodiments, the chemogenetic channel is an inhibitory chemogenetic channel, including Cation-Permeable Glycine Receptors, Ivermectin-activated Cation-Permeable Glycine Receptors, Pharmacologically Selective Actuator Modules (PSAMs), hM4Di and PSAM-GlyR. In some embodiments, the protein of interest is GFP (Green Fluorescent Protein). In some embodiments, the protein of interest is NGF (nerve growth factor), BDNF (brain-derived neurotrophic factor), or NT-3 (neurotrophin-3).

In some embodiments, the GRE is located on the sense strand of the heterologous gene coding sequence. In some embodiments, the GRE is located on the anti-sense strand of the heterologous gene coding sequence in the genome. It is within the skill of one in the art to select the appropriate sequence (e.g., GRE sequence on the sense strand, or GRE sequences on the anti-sense strand) when designing a vector using the GRE sequences as described herein. In some embodiments, the GRE is located in the expression cassette, upstream of the promoter and heterologous gene encoding the protein of interest. In some embodiments, the GRE is located in the expression cassette, about 100 bp upstream of the promoter. In some embodiments, the GRE is located in the expression cassette, about 20, about 30, about 40, about 50, about 60, about 70, about 80, about 90, about 100, about 110, about 120, about 130, about 140, about 150, about 160, about 170, about 180, about 190, or about 200 bp upstream of the promoter.

An isolated nucleic acid described herein may also contain an artificial intron, desirably located between the promoter/enhancer sequence and the protein coding sequence. In some embodiments, an intron is a synthetic or artificial (e.g., heterologous) intron. Examples of synthetic introns include an intron sequence derived from SV-40 (referred to as the SV-40 T intron sequence) and intron sequences derived from chicken beta-actin gene. In some embodiments, a heterologous gene described by the disclosure comprises one or more (1, 2, 3, 4, 5, or more) artificial introns. In some embodiments, the one or more artificial introns are positioned between a promoter and the heterologous gene encoding the protein of interest.

In some embodiments, the isolated nucleic acid comprises one or more post-transcriptional regulatory elements. Non-limiting examples of post-transcriptional regulatory elements include, but are not limited to, woodchuck hepatitis virus post-transcriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′ UTR). In some embodiments, the post-transcriptional regulatory element is a Woodchuck Hepatitis Virus Posttranscriptional Regulatory Element (WPRE).

In some embodiments, the post-transcriptional regulatory element has a sequence that has at least 80% identity to SEQ ID NO. 13. In some embodiments, the post-transcriptional regulatory element has a sequence that has at least 85% identity to SEQ ID NO. 13. In some embodiments, the post-transcriptional regulatory element has a sequence that has at least 90%, 95%, 98%, or 99% identity to SEQ ID NO. 13. In some embodiments, the post-transcriptional regulatory element has a sequence that comprises SEQ ID NO. 13. In some embodiments, the post-transcriptional regulatory element has a sequence that consists of SEQ ID NO. 13.

SEQ ID NO. 13 (WPRE):
AATCAACCTCTGGATTACAAAATTTGTGAAAGATTGACTGGTATT
CTTAACTATGTTGCTCCTTTTACGCTATGTGGATACGCTGCTTTA
ATGCCTTTGTATCATGCTATTGCTTCCCGTATGGCTTTCATTTTC
TCCTCCTTGTATAAATCCTGGTTGCTGTCTCTTTATGAGGAGTTG
TGGCCCGTTGTCAGGCAACGTGGCGTGGTGTGCACTGTGTTTGCT
GACGCAACCCCCACTGGTTGGGGCATTGCCACCACCTGTCAGCTC
CTTTCCGGGACTTTCGCTTTCCCCCTCCCTATTGCCACGGCGGAA
CTCATCGCCGCCTGCCTTGCCCGCTGCTGGACAGGGGCTCGGCTG
TTGGGCACTGACAATTCCGTGGTGTTGTCGGGGAAATCATCGTCC
TTTCCTTGGCTGCTCGCCTATGTTGCCACCTGGATTCTGCGCGGG
ACGTCCTTCTGCTACGTCCCTTCGGCCCTCAATCCAGCGGACCTT
CCTTCCCGCGGCCTGCTGCCGGCTCTGCGGCCTCTTCCGCGTCTT
CGCCTTCGCCCTCAGACGAGTCGGATCTCCCTTTGGGCCGCCTCC
CCGC

In some embodiments, the isolated nucleic acid comprises an expression cassette comprising or consisting of a mPEP1_3 (SEQ ID NO: 1), a murine CMV promoter (SEQ ID NO: 12), a heterologous gene encoding a protein of interest, WPRE (SEQ ID NO: 13) and a polyA signal (SEQ ID NO: 14), flanked with ITR sequences (SEQ ID NOs: 15-16). In some embodiments, the isolated nucleic acid comprises an expression cassette comprising or consisting of a mNP9_3 (SEQ ID NO: 3), a murine CMV promoter (SEQ ID NO: 12), a heterologous gene encoding a protein of interest, WPRE (SEQ ID NO: 13) and a polyA signal (SEQ ID NO: 14), flanked with ITR sequences (SEQ ID NOs: 15-16). In some embodiments, the protein of interest is GFP.

### III. Gene Therapy Vectors

Aspects of the present disclosure relate to gene therapy vectors (e.g., a DNA vector), comprising an isolated nucleic acid as described herein. A gene therapy vector may be a viral vector (e.g., a lentiviral vector, an adeno-associated virus vector, an adenoviral (Ad) vector, etc.) or a non-viral vector (e.g., plasmid, a closed-ended DNA (e.g., ceDNA), a lipid/DNA nanoparticle, etc.). The present disclosure also contemplates using any other delivery modalities known in the art for delivering the nucleic acids described herein, including but not limited to liquid nanoparticles (LNPs), cyclodextrin-based systems, natural polymers, Cycloamylose-based nanocarriers, Silica-based nanoparticles, etc.

In some embodiments, the viral vector is selected from a modified virus derived from a virus selected from the group consisting of a retrovirus, lentivirus, gammavirus, adenovirus, adeno-associated virus (AAV), poxvirus, alphavirus, and herpes virus. In some embodiments, the viral vector is an AAV vector, a herpes vector or a lentiviral vector. In some embodiments, the viral vector is selected from the group consisting of an AAV1 vector, an AAV2 vector, an AAV3 vector, an AAV4 vector, an AAV5 vector, an AAV6 vector, an AAV7 vector, an AAV8 vector, an AAV9 vector, an AAV PhP-S vector, an AAV10 vector, an AAV11 vector, an AAVrh.10 vector, an AAV1 R6 vector, an AAV1 R7 vector, an rAAVrh.8 vector, an AAV-BR1 vector, an AAV-PHP.B vector, an AAV-PPS vector, an AAV-PHP.eB vector, and variants thereof. In some embodiments, the viral vector is AAV PhP-S.

In some embodiments, an expression cassette comprising a GRE described herein and a heterologous gene encoding a protein of interest is flanked by one or more viral replication sequences, for example, lentiviral long terminal repeats (LTRs) or adeno-associated virus (AAV) inverted terminal repeats (ITRs).

The isolated nucleic acids of the disclosure may be recombinant adeno-associated virus (AAV) vectors (rAAV vectors). In some embodiments, an isolated nucleic acid as described by the disclosure comprises two adeno-associated virus (AAV) inverted terminal repeat (ITR) sequences, or variants thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, an expression cassette (e.g., expression cassette comprising a heterologous gene encoding a protein of interest), and 5′ and 3′ AAV inverted terminal repeats (ITRs). The isolated nucleic acids may also comprise a region encoding, for example, 5′ and 3′ untranslated regions (UTRs), and/or an expression control sequence (e.g., a polyA tail).

Generally, ITR sequences are about 145 bp in length. Preferably, substantially the entire sequence encoding the ITR is used in the isolated nucleic acid, although some degree of minor modification of these sequences is permissible. The ability to modify these ITR sequences is within the skill of one in the art. (See, e.g., texts such as Sambrook et al., Molecular Cloning. A Laboratory Manual, 2d ed., Cold Spring Harbor Laboratory, New York (1989); and K. Fisher et al., J Virol., 70:520 532 (1996)). An example of such a molecule employed in the present disclosure is an isolated nucleic acid comprising an expression cassette comprising a heterologous gene encoding a protein of interest and a GRE sequence, wherein the expression cassette is flanked by the 5′ and 3′ AAV ITR sequences. The AAV ITR sequences may be obtained from any known AAV, including presently identified mammalian AAV types. In some embodiments, the isolated nucleic acid (e.g., the rAAV vector) comprises at least one ITR having a serotype selected from AAV1, AAV2, AAV3, AAV4, AAV5, AAV6, AAV6.2, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, AAV-PHP.eB, and variants thereof. In some embodiments, the isolated nucleic acid further comprises a region (e.g., a second region, a third region, a fourth region, etc.) comprising a second AAV ITR. In some embodiments, the second AAV ITR has a serotype selected from AAV1, AAV2, AAV3, AAV4, AAV5, AAV6, AAV6.2, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, AAV-PHP.eB, and variants thereof.

In some embodiments, a gene therapy vector is a non-viral vector. In some embodiments, the non-viral vector is a plasmid, plasmid, phage, transposon, cosmid, chromosome, or artificial chromosome.

In some embodiments, the isolated nucleic acid (e.g., rAAV vector) described herein comprises a posttranscriptional response element. As used herein, the term “post-transcriptional response element” refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′ UTR). In some embodiments, the isolated nucleic acid (e.g., rAAV vector) comprises a woodchuck hepatitis virus posttranscriptional regulatory element (WPRE).

In some embodiments, the vector further comprises conventional control elements which are operably linked with elements of the heterologous gene encoding the protein of interest in a manner that permits its transcription, translation, and/or expression in a cell transfected with the vector or infected with a virus comprising the vector. Expression control sequences include appropriate transcription, initiation, termination sequences; efficient RNA processing signals, such as splicing and polyadenylation (polyA) signals; sequences that stabilize cytoplasmic mRNA; sequences that enhance translation efficiency (e.g., Kozak consensus sequence); sequences that enhance protein stability. A polyadenylation sequence generally is inserted following the coding sequences and optionally before a 3′ AAV ITR sequence. An rAAV construct useful in the disclosure may also contain an intron, desirably located between the promoter/enhancer sequence and the transgene.

In some aspects, the present disclosure provides a recombinant adeno-associated viral (rAAV) vector comprising a gene regulatory element sequence as described herein. In some embodiments, an rAAV vector described herein comprises 5′ ITR, a GRE sequence, a CMV promoter (e.g., minimal CMV promoter), a heterologous gene sequence encoding a protein of interest, a WPRE, a polyA signal, and a 3′ ITR. In some embodiments, the rAAV vector further comprises a Kozak sequence.

IV. Recombinant Adeno-Associated Viruses (rAAVs)

In some aspects, the disclosure provides isolated AAVs. As used herein with respect to AAVs, the term “isolated” refers to an AAV that has been artificially produced, engineered, or obtained. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, a rAAV having a capsid appropriate for the tissue being targeted can be selected.

Methods for obtaining recombinant AAVs having a desired capsid protein are known in the art. (See, for example, US 2003/0138772, which is incorporated herein by reference). Typically the methods involve culturing a host cell which contains a nucleic acid sequence encoding an AAV capsid protein; a functional rep gene; a recombinant AAV vector composed of AAV inverted terminal repeats (ITRs) and an expression cassette; and a helper plasmid expressing the E2b and E4 transcripts from adenovirus to permit packaging of the recombinant AAV vector into the AAV capsid. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa, and about 62 kDa. In some embodiments, upon translation, capsid proteins form a spherical 60-mer protein shell around the viral genome. In some embodiments, the functions of the capsid proteins are to protect the viral genome, deliver the genome, and/or interact with the host. In some aspects, capsid proteins deliver the viral genome to a host in a tissue specific manner (e.g., to the DRG).

The present disclosure is based in part on the finding that certain AAV serotype capsids are capable of delivering a nucleic acid comprising an expression cassette to the DRG. In some embodiments, an AAV capsid protein is of an AAV serotype selected from the group consisting of AAV1, AAV2, AAV3, AAV4, AAV5, AAV6, AAV6.2, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, AAV-PHP.eB, and variants thereof. AAV5 and AAV9 show good transduction to the DRG (Mason M R, Ehlert E M, Eggers R, et al. Comparison of AAV serotypes for gene delivery to dorsal root ganglion neurons. Mol Ther. 2010; 18(4):715-724. doi:10.1038/mt.2010.19, and Schuster D J, Dykstra J A, Riedl M S, et al. Biodistribution of adeno-associated virus serotype 9 (AAV9) vector after intrathecal and intravenous delivery in mouse. Front Neuroanat. 2014; 8:42. Published 2014 Jun. 10. doi:10.3389/fnana.2014.00042). In some embodiments, the AAV capsid is from AAV9. In some embodiments, the AAV capsid is from AAV5. In some embodiments, the AAV capsid is from AAV PhP-S.

The skilled artisan will also realize that conservative amino acid substitutions may be made to provide functionally equivalent variants or homologs of the capsid proteins. In some aspects, the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides described herein.

In some embodiments, the rAAV is a single stranded AAV (ssAAV). In some embodiments, the rAAV is a self-complementary AAV (scAAV).

In some embodiments, the rAAV as provided herein, is capable of delivering a nucleic acid comprising a GRE sequence as described herein to a mammal. In some embodiments, the mammal is a human or a non-human mammal, such as a mouse, a rat, or a non-human primate (e.g., cynomolgus monkey), a cat, a dog, a pig, a horse, a donkey, a camel, a sheep, or a goat. In certain embodiments, the mammal is a human.

Aspects of the present disclosure provide for methods of manufacturing of a rAAV virus as described herein in a host cell. The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art. A host cell may be a mammalian cell (e.g., a human cell), a yeast cell, a bacterial cell, an insect cell, a plant cell, or a fungal cell.

In some embodiments, recombinant AAVs may be produced using the triple transfection method (described in detail in U.S. Pat. No. 6,001,650, which is incorporated herein by reference). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, both of which are incorporated herein by reference. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses, such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a Laboratory Manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids into suitable host cells.

### V. Delivery of Proteins for Regulation of Nociceptor Function

The approach for the treatment of pain and neuropathies described in the present disclosure comprises the delivery, with and/or within the nucleic acid of the present disclosure, of a nucleotide sequence encoding a heterologous protein that results in the reduction of abnormal activity when expressed in peripheral nociceptors whose cell bodies are housed in the dorsal root ganglion (DRG). Any protein can be delivered. Preferably, the protein presents an interest for the treatment and/or management of a neurological disease and/or pain. A variety of different proteins are suitable for this purpose. These can be membrane proteins, i.e. proteins that become embedded in, and optionally span, the plasma membrane of neurons and/or nociceptors. Examples of membrane proteins include, but are not limited to (i) ion channels that allow passage of charged ions through the cell membrane into and out of the cell, including inhibitory ion channels such as Cl− (chlorine) or K+(potassium) ion channels, (ii) chemogenetic channels, including inhibitory chemogenetic channels that inhibit neuronal firing in response to a chemical compound or ligand that is self-administered by the patient, (iii) optogenetic channels, i.e. light-sensitive ion channels that can be operated, and therefore used to control neuron activity, through light stimulus, and (iv) custom-designed chemogenetic/optogenetic channels that a operated by a mix of chemical and light stimuli. Other, non-membrane proteins are also considered, such as pro-neurogenic peptides, or pro-neurogenic proteins, that stimulate neurogenesis and/or enhance neuron formation, such as, for example, angiopoietin-1, cannabidiol, aminopropyl carbazole or P7C3. Specific proteins can also be selected for specific peripheral neuropathies such as diabetic neuropathy or chemotherapy-induced neuropathy.

In some embodiments, the nucleic acid of the present disclosure comprises a heterologous gene encoding a protein of interest. The protein of interest can be any protein. Preferably, the protein of interest is a protein that provides a therapeutic benefit for the treatment or management of pain or a neurological disease. In some embodiments, the protein is a pro-neurogenic peptide or a pro-neurogenic protein. In some embodiments, the protein of interest is an analgesic peptide. In some embodiments, the protein of interest is an endogenous opioid, a cannabinoid, or orexin. In some embodiments, the protein is a membrane protein. In some embodiments, the membrane protein is an ion channel, a chemogenetic channel, an optogenetic channel or a custom-designed chemogenetic/optogenetic channel. In some embodiments, the ion channel is an inhibitory ion channel. In some embodiments, the inhibitory ion channel is a Kir2.1 inward-rectifier potassium ion channel. In some embodiments, the chemogenetic channel is an inhibitory chemogenetic channel, including Cation-Permeable Glycine Receptors, Ivermectin-activated Cation-Permeable Glycine Receptors, Pharmacologically Selective Actuator Modules (PSAMs), hM4Di and PSAM-GlyR. In some embodiments, the protein of interest is GFP (Green Fluorescent Protein). In some embodiments, the protein of interest is NGF (nerve growth factor), BDNF (brain-derived neurotrophic factor), or NT-3 (neurotrophin-3).

Table 2 lists sequences of exemplary heterologous genes and proteins of interest that may be used in gene therapy vectors for the treatment or management of pain. In some embodiments, the chemogenetic channel has a sequence that is at least 70% identical to any one of SEQ ID NOs: 7, 9 and 11, and is encoded by a nucleotide sequence having at least 70% identity with any one of SEQ ID NOs: 6, 8 and 10. In some embodiments, the chemogenetic channel has a sequence that is at least 75% identical to any one of SEQ ID NOs: 7, 9 and 11, and is encoded by a nucleotide sequence having at least 75% identity with any one of SEQ ID NOs: 6, 8 and 10. In some embodiments, the chemogenetic channel has a sequence that is at least 80% identical to any one of SEQ ID NOs: 7, 9 and 11, and is encoded by a nucleotide sequence having at least 80% identity with any one of SEQ ID NOs: 6, 8 and 10. In some embodiments, the chemogenetic channel has a sequence that is at least 85% identical to any one of SEQ ID NOs: 7, 9 and 11, and is encoded by a nucleotide sequence having at least 85% identity with any one of SEQ ID NOs: 6, 8 and 10. In some embodiments, the chemogenetic channel has a sequence that is at least 90% identical to any one of SEQ ID NOs: 7, 9 and 11, and is encoded by a nucleotide sequence having at least 90% identity with any one of SEQ ID NOs: 6, 8 and 10. In some embodiments, the chemogenetic channel has a sequence that is at least 95% identical to any one of SEQ ID NOs: 7, 9 and 11, and is encoded by a nucleotide sequence having at least 95% identity with any one of SEQ ID NOs: 6, 8 and 10. In some embodiments, the chemogenetic channel has a sequence that is at least 98% identical to any one of SEQ ID NOs: 7, 9 and 11, and is encoded by a nucleotide sequence having at least 98% identity with any one of SEQ ID NOs: 6, 8 and 10. In some embodiments, the chemogenetic channel has a sequence that is at least 99% identical to any one of SEQ ID NOs: 7, 9 and 11, and is encoded by a nucleotide sequence having at least 99% identity with any one of SEQ ID NOs: 6, 8 and 10. In some embodiments, the chemogenetic channel has a sequence identical to any one of SEQ ID NOs: 7, 9 and 11, and is encoded by a sequence identical to any one of SEQ ID NOs: 6, 8 and 10.

### VI. Pharmaceutical Compositions

Nucleic acids, vectors or rAAVs provided herein may be suspended in a physiologically compatible carrier (i.e., in a composition) for pharmaceutical use, and may be delivered to a subject in a pharmaceutical composition according to any appropriate method known in the art.

It should be appreciated that, in some embodiments, compositions may include one or more types of nucleic acids, vectors or rAAV provided herein. The pharmaceutical compositions of the disclosure may comprise a nucleic acid, a vector or an rAAV described herein alone, or in combination with one or more other nucleic acids, vectors or rAAVs. In some embodiments, a composition comprises 1, 2, 3, 4, 5, 6, 7, 8, 9, 10, or more different nucleic acids, vectors or rAAVs each having one or more heterologous genes encoding different proteins of interest.

In some embodiments, a pharmaceutical composition comprising an isolated nucleic acid, vector or rAAV as described herein may contain at least about 0.1%, at least about 0.5%, at least about 1%, at least about 2%, at least about 3%, at least about 4%, at least about 5%, at least about 10%, at least about 12%, at least about 15%, at least about 18%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90% of the isolated nucleic acid, vector or rAAV, or more, although the percentage of the nucleic acid may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The isolated nucleic acids, vectors or rAAVs provided herein may be formulated in any suitable manner known in the art. Generally, nucleic acids, vectors or rAAVs provided herein are formulated in a manner suitable for pharmaceutical use as described herein. For example, nucleic acids, vectors or rAAVs can be delivered to a subject using a formulation that minimizes degradation, facilitates delivery and/or uptake, and/or provides another beneficial property to the nucleic acids in the formulation. In some embodiments, provided herein are compositions comprising nucleic acids, vectors and/or rAAVs and pharmaceutically acceptable excipients. Such compositions can be suitably formulated such that when administered to a subject, either into the immediate environment of a target cell (e.g., nociceptor, cell of the central nervous system, neuron), or systemically, a sufficient amount of the nucleic acids, vectors or rAAVs enter target cells (e.g., nociceptors). In some embodiments, nucleic acids are formulated in buffered solutions, such as phosphate-buffered saline solutions, liposomes, micellar structures, and capsids, for example as described in U.S. Pat. Nos. 9,950,068, 9,050,373, 9,314,529, 8,877,237, US Patent Application Publication No. 2020/0085974 and US Patent Application Publication No. 2011/0274745, all of which are herein incorporated by reference.

In some embodiments, nucleic acids, vectors or rAAVs are formulated in water or in an aqueous solution (e.g., water with pH adjustments). In some embodiments, nucleic acids, vectors or rAAVs are formulated in a basic buffered aqueous solution (e.g., PBS) with a pH of 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, or 13.9. In some embodiments, formulations as disclosed herein comprise an excipient. In some embodiments, an excipient confers to a composition improved stability, improved absorption, improved solubility, and/or (e.g., and) therapeutic enhancement of the nucleic acid. In some embodiments, an excipient is a buffering agent (e.g., sodium citrate, sodium phosphate, a tris base, or sodium hydroxide), a sugar (e.g., lactose, sucrose, mannitol or sorbitol), a vehicle (e.g., a buffered solution, petrolatum, dimethyl sulfoxide, alcohol, or mineral oil, micelle, liposome, capsid, lipid nanoparticle), a salt, a cellulose preparation, a polymer, a lipid, an antioxidant, a preservative, a calcium phosphate, a binder (e.g., starch, gelatin, methyl cellulose, hydroxypropyl methylcellulose, sodium carboxymethylcellulose, and/or polyvinyl pyrrolidone), or any combination thereof.

In some embodiments, a nucleic acid, vector or rAAV is lyophilized for extending its shelf-life and then made into a solution before use (e.g., before administration to a subject). Accordingly, an excipient in a composition comprising a nucleic acid, vector or rAAV described herein may be a lyoprotectant (e.g., mannitol, lactose, polyethylene glycol, or polyvinyl pyrolidone) or a collapse temperature modifier (e.g., dextran, ficoll, or gelatin).

In some embodiments, rAAV compositions described herein are formulated to reduce aggregation of AAV particles in the composition, particularly where high rAAV concentrations are present (e.g., ˜10¹³ GC/ml or more). Appropriate methods for reducing aggregation may be used, including, for example, addition of surfactants, pH adjustment, salt concentration adjustment, etc. (See, e.g., Wright et al., Molecular Therapy (2005) 12, 171-178, the contents of which are incorporated herein by reference.)

In some embodiments, a pharmaceutical composition is formulated to be compatible with its intended route of administration. As used herein, the terms “administering” or “administration” means to provide a nucleic acid to a subject in a manner that is physiologically and/or (e.g., and) pharmacologically useful (e.g., to treat a condition in the subject). Examples of routes of administration include parenteral, e.g., intravenous, intradermal, subcutaneous, administration, or direct administration to the CNS (e.g., intracerebral injection, intraventricular injection, intracisternal injection, intraparenchymal injection, intrathecal injection, intracerebroventricular (ICV), injection into the cisterna magna (ICM), and any combination of the foregoing).

Pharmaceutical compositions suitable for injectable use include sterile aqueous solutions (where water soluble) or dispersions and sterile powders for the instant preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. In some embodiments, formulations include isotonic agents, for example, sugars, polyalcohols such as mannitol, sorbitol, and sodium chloride, in the composition. Sterile injectable solutions can be prepared by incorporating the nucleic acids in a required amount in a selected solvent with one or a combination of ingredients enumerated above, as required, followed by filtered sterilization to prevent the growth of microorganisms. In many cases, the form is sterile. It must be stable under the conditions of manufacture and storage and must be preserved to prevent contamination with microorganisms, such as bacteria, fungi, and other viruses. Proper fluidity may be maintained, for example, by the use of a coating, such as lecithin, by the maintenance of the required particle size in the case of dispersion and by the use of surfactants. The prevention of contamination by microorganisms can be achieved by various antibacterial and antifungal agents, for example, parabens, chlorobutanol, phenol, sorbic acid, thimerosal, and the like. In many cases, it will be preferable to include isotonic agents, for example, sugars or salts (e.g., sodium chloride). Prolonged absorption of the injectable composition can be achieved by the use in the composition of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous administration, intramuscular administration, subcutaneous administration, intraperitoneal administration, and injection through the round window membrane of the inner ear. In this respect, a suitable sterile aqueous medium may be employed. For example, one dosage may be dissolved in 1 ml of isotonic NaCl solution and either added to 1000 ml of hypodermoclysis fluid or injected at the proposed site of infusion (see for example, Remington's Pharmaceutical Sciences 15th Edition, pages 1035-1038 and 1570-1580). Some variation in dosage will necessarily occur depending on the condition of the host. The person responsible for administration will, in any event, determine the appropriate dose for the individual subject/host.

Sterile injectable solutions are prepared by incorporating the active isolated nucleic acid, vector or rAAV in the required amount in the appropriate solvent with various of the other ingredients described herein, as required, followed by filter sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may also be formulated in a neutral or salt form. Pharmaceutically-acceptable salts include but are not limited to hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for the introduction of the compositions of the present disclosure into suitable host cells. In particular, the isolated nucleic acids, vectors or rAAVs may be formulated for delivery either encapsulated in a lipid particle, a liposome, a vesicle, a nanosphere, a nanoparticle, or the like. In some embodiments, the pharmaceutical composition comprises a lipid microparticle, a lipid nanoparticle, a polymeric microparticle, a polymeric nanoparticle, a liposome, an endosome, a micelle, a vesicle, a gold nanoparticle, a carbon nanotube, a quantum dot, a magnetic nanoparticle, a dendrimer, a calcium phosphate vehicle, a DNA-coated microparticle or a polymer. In some embodiments, the composition further comprises a pharmaceutically acceptable carrier.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516, which is incorporated herein by reference). Further, various methods of liposome and liposome-like preparations as potential drug carriers have been described (U.S. Pat. Nos. 5,567,434; 5,552,157; 5,565,213; 5,738,868 and 5,795,587, each of which is incorporated herein by reference).

Alternatively, nanocapsule formulations of composition may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In some embodiments, the pharmaceutical composition may be in solid form, aqueous form, or a liquid form. In some embodiments, an aqueous or liquid form may be lyophilized. In some embodiments, a lyophilized form may be reconstituted with an aqueous or liquid solution. In some embodiments, a pharmaceutical composition may further comprise any other suitable therapeutic agent for treatment of a subject, e.g., a human subject having neurological disease or condition such as pain, back pain, chronic pain, a peripheral neuropathy, a diabetic neuropathy, neuralgia, a chemotherapy-induced neuropathy, migraine or trigeminal neuralgia. In some embodiments, the other therapeutic agents may enhance or supplement the effectiveness of the nucleic acids, vectors or rAAVs described herein. In some embodiments, the other therapeutic agents may function to treat a different symptom or disease than the nucleic acids, vectors or rAAVs described herein. In some embodiments, the pharmaceutical composition is for use in treating or slowing the progression and/or reversing a neurological disease or condition such as pain, back pain, chronic pain, a peripheral neuropathy, a diabetic neuropathy, neuralgia, a chemotherapy-induced neuropathy, migraine or trigeminal neuralgia. In some embodiments, the subject is a non-human primate or rodent. In some embodiments, the subject is a human.

### VII. Experimental and Therapeutic Applications

In some aspects, the present disclosure also provides a method for selectively expressing a protein of interest in a peptidergic or non-peptidergic nociceptor, comprising contacting the nociceptor with a nucleic acid described herein, a vector described herein, an rAAV described herein, or a pharmaceutical composition described herein.

The isolated nucleic acids, vectors or rAAVs, preferably in a pharmaceutical composition as described herein, may be administered to a subject, e.g., host animal, patient, experimental animal. In some embodiments, the subject is a mammal. In some examples, the mammal is a human. In other embodiments, the mammal can be a non-human mammal, such as a human, rodent, mouse, rat, cat, dog, sheep, rabbit, horse, cow, goat, pig, guinea pig, hamster, chicken, turkey, or a non-human primate (e.g., cynomolgus monkey). The subject may be at any stage of development and of any gender.

Aspects of the disclosure include a method comprising administering to a subject an effective amount of a nucleic acid, vector, rAAV or composition as described herein. In some embodiments, an effective amount of a pharmaceutical composition that comprises a nucleic acid, vector or rAAV can be administered to a subject in need of treatment. In some aspects, the present disclosure also provides a method of treating a neurological disease or condition in a subject comprising administering to the subject a nucleic acid described herein, a vector described herein, an rAAV described herein, or a pharmaceutical composition described herein. In some embodiments, the administering results in a reduction of abnormal activity of nociceptors. In some embodiments, the neurological disease or condition is pain, back pain, chronic pain, a peripheral neuropathy, a diabetic neuropathy, neuralgia, a chemotherapy-induced neuropathy, migraine or trigeminal neuralgia.

The pharmaceutical composition can be delivered to any organ or tissue of interest. In some embodiments, the pharmaceutical composition is delivered to the DRG. The nucleic acids, vectors or rAAVs are administered in sufficient amounts to transfect the cells of the desired tissue (e.g., the DRG) and to provide sufficient levels of gene transfer and expression without undue adverse effects. The dose of nucleic acids, vectors or rAAVs required to achieve a particular “therapeutic effect,” will vary based on several factors including, but not limited to: the route of administration, the level of gene or RNA expression required to achieve a therapeutic effect, the specific disease or disorder being treated, and the stability of the nucleic acid, vector or rAAV. One of skill in the art can readily determine a rAAV virion dose range to treat a patient having a particular disease or disorder (e.g., neurological disease) based on the aforementioned factors, as well as other factors.

An effective amount of a nucleic acid, vector or rAAV described herein is an amount sufficient to infect an animal (e.g., mouse, rat, non-human primate or human) or target a desired tissue or cell (e.g., peptidergic and non-peptidergic nociceptors). The effective amount will depend primarily on factors, such as the species, age, weight, health of the subject, and the tissue to be targeted, and may thus vary among animals and tissue. For example, an effective amount of the rAAV is generally in the range of from about 1 ml to about 100 ml of solution containing from about 10⁹ to 10¹⁶ genome copies. In some cases, a dosage between about 10¹¹ to 101 rAAV genome copies is appropriate. In certain embodiments, 10⁹ rAAV genome copies are effective to target inner ear tissue (e.g., connective tissue cells of the cochlea and supporting cells of the organ of Corti and nearby regions). In some embodiments, a dose more concentrated than 10⁹ rAAV genome copies is toxic when administered to the ear of a subject. In some embodiments, an effective amount is produced by multiple doses of a rAAV.

In some embodiments, a dose of isolated nucleic acid, vector or rAAV described herein is administered to a subject no more than six times per day (e.g., every four hours). In some embodiments, a dose of isolated nucleic acid, vector or rAAV described herein is administered to a subject no more than four times per day (e.g., every six hours). In some embodiments, a dose of isolated nucleic acid, vector or rAAV described herein is administered to a subject no more than twice per day (e.g., every 12 hours). In some embodiments, a dose of isolated nucleic acid, vector or rAAV described herein is administered to a subject no more than once per day (e.g., a 24-hour period). In some embodiments, a dose of nucleic acid, vector or rAAV is administered to a subject no more than once per 2, 3, 4, 5, 6, or 7 days. In some embodiments, a dose of nucleic acid, vector or rAAV is administered to a subject no more than once per week (e.g., 7 calendar days). In some embodiments, a dose of nucleic acid, vector or rAAV is administered to a subject no more than bi-weekly (e.g., once in a two-week period). In some embodiments, a dose of nucleic acid, vector or rAAV is administered to a subject no more than once per month (e.g., once in 30 calendar days). In some embodiments, a dose of nucleic acid, vector or rAAV is administered to a subject no more than once per six months. In some embodiments, a dose of rAAV is administered to a subject no more than once per year (e.g., 365 days or 366 days in a leap year). In some embodiments, a dose of nucleic acid, vector or rAAV is administered to a subject once in a lifetime.

In some embodiments, a pharmaceutical composition that comprises a nucleic acid, vector or rAAV described herein is administered at an effective concentration that confers a therapeutic effect on the subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of a nucleic acid, vector or rAAV described herein in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of pain symptoms associated with AD, through measures of a subject's self-reported sensitivity to external stimuli, e.g. mobility, self-care, usual activities, pain/discomfort, or other wellness and quality-of-life indicators.

In some embodiments, a pharmaceutical composition that comprises a nucleic acids, vectors or rAAVs described herein is administered to a subject at an effective concentration sufficient to inhibit abnormal activity of nociceptors, or reduce perceived pain levels, by at least 10%, at least 20%, at least 30%, at least 40%, at least 50%, at least 60%, at least 70%, at least 80%, at least 90%, or at least 95% relative to a control, e.g. baseline level of nociceptor activity, or perceived pain, prior to treatment, or compared to a control population.

In some embodiments, a single dose or administration of a pharmaceutical composition that comprises a nucleic acid, vector or rAAV described herein to a subject is sufficient to inhibit abnormal nociceptor activity and/or reduce pain levels for at least 1-5, 1-10, 5-15, 10-20, 15-30, 20-40, 25-50, or more days. In some embodiments, a single dose or administration of a pharmaceutical composition that comprises a nucleic acid, vector or rAAV described herein to a subject is sufficient to inhibit abnormal nociceptor activity and/or reduce pain levels for at least 1, 2, 3, 4, 5, 6, 7, 8, 9, 10, 11, 12, 15, 20, or 24 weeks. In some embodiments, a single dose or administration of a pharmaceutical composition that comprises a nucleic acid, vector or rAAV described herein to a subject is sufficient to inhibit abnormal nociceptor activity and/or reduce pain levels for at least 1-5, 1-10, 2-5, 2-10, 4-8, 4-12, 5-10, 5-12, 5-15, 8-12, 8-15, 10-12, 10-15, 10-20, 12-15, 12-20, 15-20, or 15-25 weeks. In some embodiments, a single dose or administration of a pharmaceutical composition that comprises a nucleic acid, vector or rAAV described herein to a subject is sufficient to inhibit abnormal nociceptor activity and/or reduce pain levels for at least 1, 2, 3, 4, 5, or 6 months.

In some embodiments, a pharmaceutical composition comprising a nucleic acid, vector or rAAV as described herein may be administered by a suitable route, which may include a bolus injection or a continuous infusion over a period of time. Examples of pharmaceutically acceptable routes of administration include, but are not limited to, direct delivery to the selected organ or tissue (e.g., the DRG or the cisterna magna), intravenous, intramuscular, subcutaneous, intradermal, intratumoral, intracerebral injection, intracerebroventricular, intraventricular, intracisternal, intraparenchymal, intrathecal injection, and any combination of the foregoing and other parenteral routes of administration. In some embodiments, the administering to a subject is through injection. In some embodiments, the injection comprises In some embodiments, the injection comprises an intracerebroventricular (ICV) injection, an intrathecal injection, an intravenous (IV) injection, or an injection into the cisterna magna (ICM).

### VIII. Kits and Related Compositions

The nucleic acids, vectors, or rAAVs described herein may, in some embodiments, be assembled into pharmaceutical or research kits to facilitate their use in therapeutic or research applications. A kit may include one or more containers housing the nucleic acids of the disclosure and instructions for use. Specifically, such kits may include one or more nucleic acids described herein, along with instructions describing the intended application and the proper use of these nucleic acids. In certain embodiments, agents in a kit may be in a pharmaceutical formulation and dosage suitable for a particular application and for a method of administration or usage of the nucleic acids, as described herein. Kits for research purposes may contain the components in appropriate concentrations or quantities for performing various experiments.

In some embodiments, the instant disclosure relates to a kit for administering a nucleic acid as described herein. In some embodiments, the kit comprising a container housing the nucleic acid, and devices (e.g., syringe) for extracting the nucleic acid from the container. In some embodiments, the device for extracting the nucleic acids from the container is also used for administration (e.g., injection).

In some embodiments, the instant disclosure relates to a kit for producing a nucleic acid.

In some embodiments, the instant disclosure relates to a kit for use in treating pain, back pain, chronic pain, a peripheral neuropathy, a diabetic neuropathy, neuralgia, a chemotherapy-induced neuropathy, migraine or trigeminal neuralgia. In some embodiments, the kit is for inhibiting the abnormal function of nociceptors.

The kit may be designed to facilitate practicing the methods described herein by researchers, clinicians or healthcare professionals and can take many different forms. Each of the compositions of the kit, where applicable, may be provided in liquid form (e.g., in solution) or in solid form (e.g., a dry powder). In certain cases, some of the compositions may be reconstitutable or otherwise processable (e.g., to an active form), for example, by the addition of a suitable solvent or other medium (for example, water or a cell culture medium), which may or may not be provided in the kit. As used herein, “instructions” can include a component of instruction and/or promotion, and typically involve written instructions on or associated with the packaging. Instructions also can include any oral or electronic instructions provided in any manner such that a user will clearly recognize that the instructions are to be associated with the kit, for example, audiovisual (e.g., videotape, DVD, CD-ROM, website links for downloadable file, etc.), Internet, and/or web-based communications, etc. The written instructions may be in a form prescribed by a governmental agency regulating the manufacture, use, or sale of pharmaceuticals or biological products, which instructions can also reflect approval by the agency of manufacture, use, or sale for human or animal administration.

The kit may contain any one or more of the components described herein in one or more containers. As an example, in one embodiment, the kit may include instructions for mixing one or more components of the kit and/or isolating and mixing a sample and administering it to a subject. The kit may include a container housing a nucleic acid described herein. The nucleic acid may be in the form of a liquid, gel, or solid (e.g., powder). The nucleic acid may be prepared sterilely, packaged in a syringe, and shipped refrigerated. Alternatively, the nucleic acid may be housed in a vial or other container for storage. A second container may have other pharmaceutical agents or excipients prepared sterilely. Alternatively, the kit may include the nucleic acid premixed and shipped in a syringe, vial, tube, or other container.

### IX. General Techniques

The practice of the present disclosure will employ, unless otherwise indicated, conventional techniques of molecular biology (including recombinant techniques), microbiology, cell biology, biochemistry, and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995)

Without further elaboration, it is believed that one skilled in the art can, based on the present disclosure, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Exemplary embodiments of the present disclosure will be described in more detail by the following examples. These embodiments are exemplary of the disclosure, which one skilled in the art will recognize is not limited to the exemplary embodiments.

### EXAMPLES

### Example 1: Genomic Regulatory Elements (GREs) that Drive Viral Payload Expression in Nociceptors

In order to increase the safety, specificity and efficacy of gene therapy for the treatment of pain, it is important to target the delivery of the genetic treatment to the specific cells that are causing the pain condition. The present disclosure provides genomic regulatory elements that are capable of driving strong and restricted viral payload expression in peptidergic and non-peptidergic nociceptors with minimal expression in other DRG cell types. Using these regulatory elements, novel AAVs are designed and tested to evaluate their expression in various cell types of the DRG, and their performance in reducing abnormal activity of nociceptors.

### Example 2: Expression of GFP Reporter in the Mouse DRG in the Presence of mPEP1_3

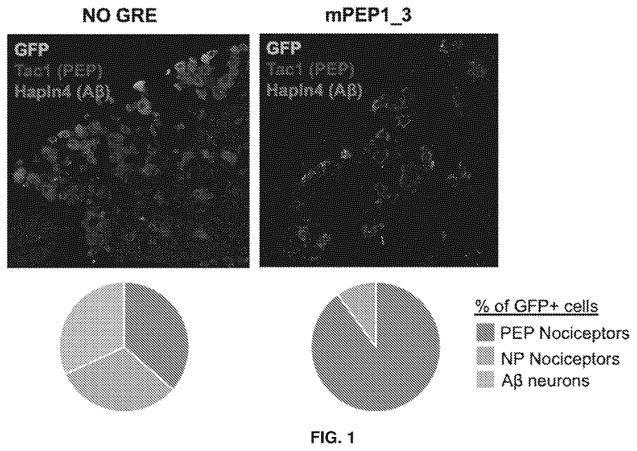

Neonatal mice received an intracerebroventricular injection of AAV vectors comprising a nucleotide sequence comprising two AAV ITRs flanking: (i) a mouse PEP1_3 (mPEP1_3, SEQ ID NO: 1), (ii) a mouse CMV promoter, (iii) a sequence encoding a green fluorescent protein (GFP) reporter, (iv) a Woodchuck Hepatitis Virus Posttranscriptional Regulatory Element (WPRE), and (v) a polyA signal sequence. A control group was injected with the same AAV vectors lacking the mPEP1_3 sequence. Fluorescence was measured in DRG cells with confocal microscopy three weeks after the injection. In the control group, fluorescence levels were observed in nociceptors as well as in Aβ neurons (FIG. 1, left side). When the injected AAV vectors comprised mPEP1_3, fluorescence was detected in nociceptors (FIG. 1, right side) but was not significant in other cell types. These results show that the mPEP1_3 sequence directs viral payload expression specifically in nociceptors, which can be leveraged to target the treatment of pain and neuropathies to these cell types.

### Example 3: Expression of GFP Reporter in the Mouse DRG in the Presence of mNP9_3

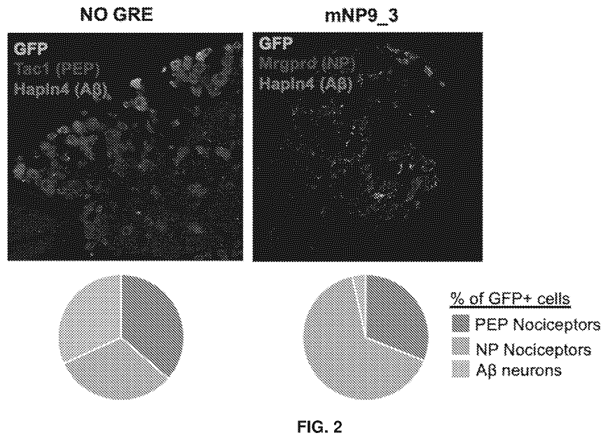

Neonatal mice received an intracerebroventricular injection of AAV vectors comprising a nucleotide sequence comprising two AAV ITRs flanking: (i) a mouse NP9_3 (mNP9_3, SEQ ID NO: 3), (ii) a mouse CMV promoter, (iii) a sequence encoding a GFP reporter protein, (iv) a WPRE sequence, and (v) a polyA signal sequence. A control group was injected with the same AAV vectors lacking the mNP9_3 sequence. Fluorescence was measured in DRG cells with confocal microscopy three weeks after the injection. In the control group, fluorescence levels were observed in nociceptors as well as in Aβ neurons (FIG. 2, left side). When the injected AAV vectors comprised mNP9_3, fluorescence was detected in nociceptors at much higher levels than in other cell types (FIG. 2, right side). These results show that the mNP9_3 sequence confers a higher specificity of viral payload expression in nociceptors, compared to other cells of the DRG. This property can be leveraged to target the treatment of pain and neuropathies to nociceptors.

### Example 4: Expression of GFP Reporter in the Mouse DRG in the Presence of PEP1_17

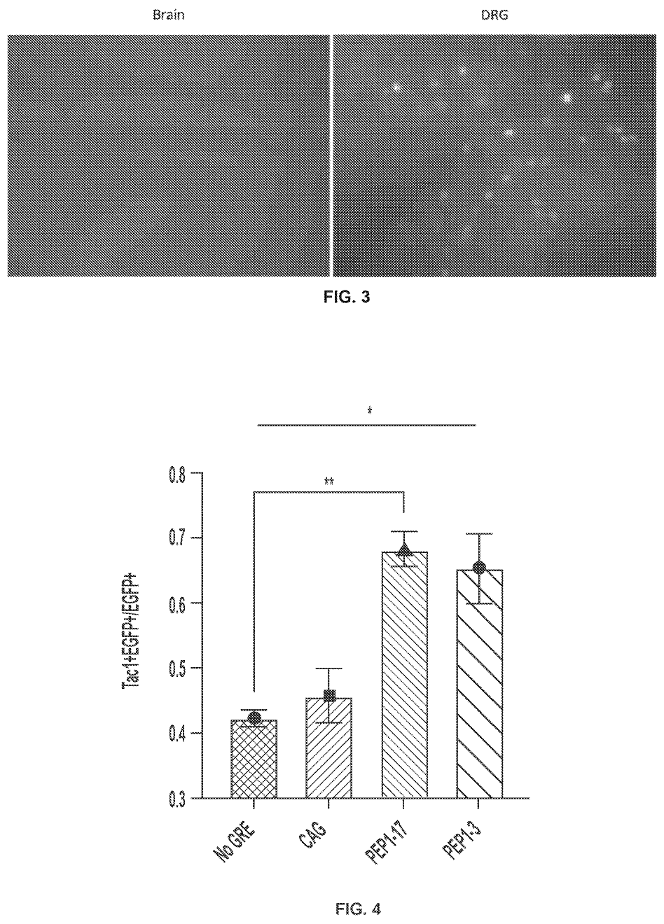

Neonatal mice received an intracerebroventricular injection of AAV vectors comprising (i) a nucleotide sequence encoding a GFP reporter protein and (ii) a PEP1_17 sequence (SEQ ID NO: 5). Fluorescence was measured in the brain and the DRG with confocal microscopy three weeks after the injection. GFP expression is observed in small diameter DRG neurons, which are enriched for nociceptors, but not significantly in brain cells (FIG. 3). These results show that the PEP1_17 sequence drives viral payload expression specifically in nociceptors within the DRG, as compared to brain cells.

The percentage of GFP+ nociceptors (Tac1+ cells) within the EGFP positive cells in the DRG were quantified, and the results suggests that mice received the AAV vectors carry either the PEP1_17 and PEP1_3 GREs increased the percentage of EGFP positive nociceptors significantly as compared to mice received AAV vector that did not carry any GRE or AAV vector having a CAG promoter for driving EGFP expression. These data demonstrates that the inclusion of the GREs in the AAV vector were able to restrict transgene expression in nociceptors in the DRG.

TABLE 1
Sequences encoding gene regulatory elements
(GREs) described in the present disclosure.

| SEQ | | |
|---|---|---|
| ID | Name/ | |
| NO | Description | Sequence |
| 1 | Mouse | TAATGAGGGCCCTGTAACTCGAGTCAGAGC |
| | PEP1_3 | ATGTAATTCTCTTCCTAGGTTGCTCTCCAG |
| | (mPEP1_3) | CTAGACTTTTATGACTTTAAGTACTTCATG |
| | | ATCAGTAAATAATAAATTCAATTTACCAGA |
| | | TAGAAGTTTTGTCACTTAGAAACACTCACT |
| | | TGGAATATATATTTCTTAATTGGCATCAGT |
| | | GGCACATTTCCCAGGAAGCATAACAAAACG |
| | | GATCATTTAATAGATGCCCAATCTCTCCAT |
| | | CCATCTATCTATCAGTGCGGTACATGATGT |
| | | TTTGACTTTAATATGGTAATTATCTCAGGC |
| | | TGACTTCAGAGCAGGAAGCTGCCTCCCACA |
| | | CAGCAGTGCTTAATTACTTTAGACTTCAGC |
| | | TTGCCAAGTGAAAAATTGACTGTTTTTTTG |
| | | CACATTGCATGGAATCGAAATGATTTCATT |
| | | GTCATTTCAGCTGAGATACAGCCTGACATC |
| | | TGCATAGAATCACGGCAAAGATAATGCAGA |
| | | ATTTCTCTGAAATTCTAAAATGGCCTGAAA |
| | | AGCTGCCTTATTTTATT |
| 2 | Human | AGGCAGCTTTCAGACCATTTTAGAATTTCA |
| | PEP1_3 | GAGGAATTTTGCATTATCTTTCCCATGATT |
| | | CTATGCTGATGTCAGGCTGTATTTCAACTG |
| | | AAATGACAATGAAATCATTTCAATTCCATG |
| | | CAATGTGCAAAAAAACAGTCAATTTTTCAC |
| | | TTGGCAAGCTGAAGTCTAAAGTAATTAAGC |
| | | ACTGCTGTGTGGGAGGCAGCTTCCTGCTCT |
| | | GAAGTCAGCCTGAGATAATTACCATATTAA |
| | | AGTCAAAACATCATGTGCCGTGCTGATAGA |
| | | TAGATGGATGGAGAGATTGGGCATCTATTA |
| | | AATGATCCATTTTGTTATGCTTCCTGGGAA |
| | | ATGTGCCATTGATGCCAATTAAGAAATATA |
| | | TATTCCAAGTGAGTATTTCTAAGTGGAAAA |
| | | ACTTCTATCTGGTAAATTGAATTTATTATT |
| | | TACTGATCATGAAGTACTTAAAGTCATAAA |
| | | ATCTAGATAGAGAGCAACCTATGAGGAGAA |
| | | TTACATGCTCTGTCTTGGTTTACAAACCCC |
| | | TCATTA |
| 3 | Mouse | CTGTACCCGGTAGGAGTGCTTTTGCCATCA |
| | NP9_3 | GTAAGAGGAAGAATCCTGGCGATGTTTCCA |
| | (mNP9_3) | GGGAAAGGAAAGGAGAACAGAAAAGGTGGG |
| | | GCCTCCAATCAGGCCAGAGAGGGTGAGGCA |
| | | GAGCCATCTCCACTTCCTTTGTGCGTCTCC |
| | | TAGAATCACCCCCCTCCCATTCCAATCTCC |
| | | TGTGGCTTTTACAGATAAGGGTTTCTCTCC |
| | | CCCTTTCTCACTCTCCATCTCTCCCAGGTT |
| | | CACTGGAGGCCTCTGAGGGCTTGAGATAGG |
| | | TTTTTCTTTGCATAAGCCAAACTTCTACTT |
| | | AACCCAGTGAGTGCTCTTATGCAGAAGGTA |
| | | CCTTTAAGTGATCACCTGACTGAAGCGAAT |
| | | GTGATGTTGCTTATCCTTTTATCAAAACAT |
| | | GGACCAATTCCCC |
| 4 | Human | GGAAAATCAGTGCAGGTTTCCAGAAGAGGA |
| | NP9_3 | TGTGACAACTCACATTTTGTTCAATTGAGT |
| | | TAAATAAGCACTTATAGAGTGTCTACCACA |
| | | TGCAGAACACTCACTGAATCGAGTATGGAT |
| | | GATACAAAGAAAAATCTATCCCAAGCCCTC |
| | | CTGCCTACACTGCCTGAGCCTCATGGGACC |
| | | CTAAATAAGTTTGGTAAGGAATGAGTGGTG |
| | | AGAAAGGGTGAGGAGAAACAAGAGCACACA |
| | | TCGTCTTGATACGGGATCTAAATGGGTGCA |
| | | TGGTTCTAGGAGAGGCACAAGGGTTTATGG |
| | | AGGTAGAGATTCCCAGTGTCCCAGATTTGT |
| | | TTCTTCACTCTCAGGTCAAAGCAGGCCTTA |
| | | TTTAGGAGGCCCAGTCATTCCTCATTTCCC |
| | | TTCCTTGGTAATATCACTACGATTCTCTCT |
| | | CCTCCCACTGGCCAAAGTGTTTGATTAAGC |
| | | CTGCAG |
| 5 | PEP1_17 | ACAGTGGTCTCAGCATTCGGTCAGCTGTGA |
| | | GGAGGCCTTGCGAGGCTCCAGGCCCTGCAC |
| | | TTCCCACCAGCATTTGGCGTCCTTGTGTTT |
| | | AATTTGGACCATGCACACGTGTGACACCCC |
| | | AGTGTGGTTGTGGTCACCGATTCATGATGA |
| | | CCCCTCTTTCAGTGTTTATTGTTCTGTCCG |
| | | TGTCTTGCTCTCTGAAGACTCAGTGAGATT |
| | | CGCATTGGAATATTTGCGGTTTTATTGAAG |
| | | AGAAGAGCTTGGGGTCTTTGACAGACGTGT |
| | | GTTAGGAACATTTCTCCATCTGCGGTTGCC |
| | | ATTTACTTTTTAATTGCATCTTCTGATGAT |
| | | TGTTTTAAATTTTAATGACGTCTAATTTGC |
| | | CCAGTTTTTCTTCTGTGGTTAGTTTGTCCC |
| | | AAACACATGGTGGGGCACTCCTGATGTCTG |
| | | CTTTGAGTCAGTACATTGCAGGTTTGATAT |
| | | GCATGTCTCACACTGTGCCGTGTGGCTCTA |
| | | TTTTCTTTAACTGGCACACAGTCCTTCTGA |
| | | CATCCTACTGTCATTTCTTGGGGAACGTTA |
| | | TGCTTCTCTCTGAGTAACTGTGGTCCCTTG |
| | | TGAGTACCAGTGCTGGTGGGCGGTGGCCTT |
| | | CTTCTTCACTCC |

TABLE 2
Nucleotide and amino acid sequences
encoding exemplary heterologous
proteins described in the present
disclosure.

| SEQ | Name/ | |
|---|---|---|
| ID | Descrip- | |
| NO | tion | Sequence |
| 6 | hGlyR | ATGCGCTGTTCTCCAGGCGGCGTGTGGCTC |
| | nucleotide | GCCCTGGCTGCTTCCCTTCTGCACGTTAGC |
| | sequence | CTGCAGGGTGAGTTCCAGCGCAAACTGTAT |
| | | AAGGAGCTTGTTAAGAATTATAACCCCCTG |
| | | GAGCGGCCGGTCGCAAATGATTCCCAGCCA |
| | | CTGACAGTGTACTTCAGCCTCTCCTTGCTG |
| | | CAGATCATGGACGTGGATGAAAAGAACCAG |
| | | GTGCTGACCACTAATATTTGGTTGCAGATG |
| | | TCCTGGACCGATCACTACTTGCAGTGGAAT |
| | | GTGAGCGAATACCCAGGTGTAAAGACTGTA |
| | | AGATTCCCTGACGGCCAAATCTGGAAACCA |
| | | GATATCCTGCTGTACAACAGCGCAGACGAA |
| | | AGGTTTGATGCAACATTTCACACCAACGTG |
| | | GGAGTCAATTCTTCAGGCCACTGCCTGTAC |
| | | CTGCCCCCTGGAATCTTCAAGTCCTCATGC |
| | | TATATCGACGTCCGCTGGTTTCCCTTCGAC |
| | | GTCCAGCACTGCAAACTCAAATTCGGGAGC |
| | | TGGAGCTACGGCGGATGGAGCCTGGATCTG |
| | | CAAATGCAGGAGGCTGACATCTCTGGTTAC |
| | | ATCCCGAATGGGGAGTGGGACCTTGTGGGA |
| | | ATCCCCGGTAAAAGAAGCGAGCGATTTTAT |
| | | GAATGCTGCAAAGAGCCCTTCCCAGATGTC |
| | | ACCTTCACAGTGACCATGCGGAGACGCATG |
| | | GGTTATTATCTGATCCAAATGTATATCCCA |
| | | AGCTTGCTTATAGTGATTTTGTCATGGATC |
| | | TCCTTCTGGATTAATATGGACGCCGCTCCA |
| | | GCTAGGGTCGGACTGGGCATCACCACAGTG |
| | | CTGACAATGACTACTCAGAGCTCAGGCAGC |
| | | CGAGCCAGCTTGCCCAAGGTTTCTTACGTG |
| | | AAGGCCATCGATATCTGGATGGCTGTCTGC |
| | | CTTCTGTTTGTCTTCAGCGCACTGCTGGAA |
| | | TACGCCGCTGTCAATTTTGTGTCTCGACAG |
| | | CATAAAGAGCTGTTGCGGTTCAGAAGAAAA |
| | | CGACGCCACCACAAAGAGGATGAGGCAGGA |
| | | GAAGGACGCTTCAACTTTAGCGCCTATGGT |
| | | ATGGGACCTGCTTGCCTCCAGGCTAAAGAC |
| | | GGAATTTCCGTGAAGGGAGCCAACAATAGC |
| | | AACACAACCAACCCACCCCCTGCTCCATCT |
| | | AAGAGCCCGGAGGAAATGCGCAAACTCTTT |
| | | ATTCAGAGAGCGAAAAAGATCGACAAAATC |
| | | TCCCGGATCGGATTCCCCATGGCTTTCCTG |
| | | ATTTTCAACATGTTTTATTGGATCATCTAC |
| | | AAGATTGTGCGAAGGGAGGACGTACACAAC |
| | | CAGTAA |
| 7 | hGlyR | MRCSPGGVWLALAASLLHVSLQGEFQRKLY |
| | protein | KELVKNYNPLERPVANDSQPLTVYFSLSLL |
| | sequence | QIMDVDEKNQVLTTNIWLQMSWTDHYLQWN |
| | | VSEYPGVKTVRFPDGQIWKPDILLYNSADE |
| | | RFDATFHTNVGVNSSGHCLYLPPGIFKSSC |
| | | YIDVRWFPFDVQHCKLKFGSWSYGGWSLDL |
| | | QMQEADISGYIPNGEWDLVGIPGKRSERFY |
| | | ECCKEPFPDVTFTVTMRRRMGYYLIQMYIP |
| | | SLLIVILSWISFWINMDAAPARVGLGITTV |
| | | LTMTTQSSGSRASLPKVSYVKAIDIWMAVC |
| | | LLFVFSALLEYAAVNFVSRQHKELLRFRRK |
| | | RRHHKEDEAGEGRFNFSAYGMGPACLQAKD |
| | | GISVKGANNSNTTNPPPAPSKSPEEMRKLF |
| | | IQRAKKIDKISRIGFPMAFLIFNMFYWIIY |
| | | KIVRREDVHNQ* |
| 8 | PSAM1 | ATGCGCTGTTCTCCAGGCGGCGTGTGGCTC |
| | nucleotide | GCCCTGGCTGCTTCCCTTCTGCACGTTAGC |
| | sequence | CTGCAGGGTGAGTTCCAGCGCAAACTGTAT |
| | | AAGGAGCTTGTTAAGAATTATAACCCCCTG |
| | | GAGCGGCCGGTCGCAAATGATTCCCAGCCA |
| | | CTGACAGTGTACTTCAGCCTCTCCTTGCTG |
| | | CAGATCATGGACGTGGATGAAAAGAACCAG |
| | | GTGCTGACCACTAATATTTGGTTGCAGATG |
| | | TCCTGGACCGATCACTACTTGCAGTGGAAT |
| | | GTGAGCGAATACCCAGGTGTAAAGACTGTA |
| | | AGATTCCCTGACGGCCAAATCTGGAAACCA |
| | | GATATCCTGCTGTACAACAGCGCAGACGAA |
| | | AGGTTTGATGCAACATTTCACACCAACGTG |
| | | GGAGTCAATTCTTCAGGCCACTGCCTGTAC |
| | | CTGCCCCCTGGAATCTTCAAGTCCTCATGC |
| | | TATATCGACGTCCGCTGGTTTCCCTTCGAC |
| | | GTCCAGCACTGCAAACTCAAATTCGGGAGC |
| | | TGGAGCTACGGCGGATGGAGCCTGGATCTG |
| | | CAAATGCAGGAGGCTGACATCTCTGGTTAC |
| | | ATCCCGAATGGGGAGTGGGACCTTGTGGGA |
| | | ATCCCCGGTAAAAGAAGCGAGCGATTTTAT |
| | | GAATGCTGCAAGGAACCCTTCCCTGACGTA |
| | | ACATTCACAGTTATCATCAGAAGAAGGCCA |
| | | TTGTTCTACGCCGTTAGTTTGTTGCTCCCC |
| | | AGTATTTTTCTCATGGTCGTGGACATCGTG |
| | | GGATTTTGTCTCCCACCTGATAGCGGGGAG |
| | | AGGGTCTCCTTTAAGATTACCTTGTTGCTC |
| | | GGCTATTCTGTATTTCTGATCATCGTGTCC |
| | | GATACCCTTCCTGCCACAATCGGCACTCCG |
| | | CTGATAGGAGTGTATTTCGTCGTGTGTATG |
| | | GCACTCCTGGTGATAAGTCTGGCGGAAACT |
| | | ATCTTCATTGTACGGCTGGTACATAAGCAG |
| | | GACCTGCAAAGACCCGTGCCAGACTGGTTG |
| | | CGACACCTTGTGCTGGACAGAATTGCATGG |
| | | ATTCTGTGTCTTGGCGAGCAACCTATGGCC |
| | | CACCGGCCACCTGCAACCTTTCAAGCCAAC |
| | | AAGACAGACGATTGTAGTGGGTCTGATCTG |
| | | TTGCCTGCTATGGGGAATCACTGCTCCCAT |
| | | GTTGGGGGACCACAAGATTTGGAAAAGACC |
| | | CCACGGGGGCGGGGATCACCCCTTCCTCCT |
| | | CCCCGAGAAGCCTCTCTCGCTGTCCGGGGG |
| | | CTGCTCCAGGAACTGTCAAGCATCCGACAT |
| | | TTTCTGGAGAAGCGGGACGAGATGCGCGAA |
| | | GTCGCTCGCGACTGGCTGCGAGTGGGCTAC |
| | | GTCCTTGACAGGCTGCTGTTTCGGATCTAC |
| | | TTGCTGGCGGTGCTGGCTTATTCCATTACT |
| | | CTGGTGACACTCTGGTCCATATGGCACTAC |
| | | AGTTA |
| 9 | PSAM1 | MRCSPGGVWLALAASLLHVSLQGEFQRKLY |
| | protein | KELVKNYNPLERPVANDSQPLTVYFSLSLL |
| | sequence | QIMDVDEKNQVLTTNIWLQMSWTDHYLQWN |
| | | VSEYPGVKTVRFPDGQIWKPDILLYNSADE |
| | | RFDATFHTNVGVNSSGHCLYLPPGIFKSSC |
| | | YIDVRWFPFDVQHCKLKFGSWSYGGWSLDL |
| | | QMQEADISGYIPNGEWDLVGIPGKRSERFY |
| | | ECCKEPFPDVTFTVIIRRRPLFYAVSLLLP |
| | | SIFLMVVDIVGFCLPPDSGERVSFKITLLL |
| | | GYSVFLIIVSDTLPATIGTPLIGVYFVVCM |
| | | ALLVISLAETIFIVRLVHKQDLQRPVPDWL |
| | | RHLVLDRIAWILCLGEQPMAHRPPATFQA |
| | | NKTDDCSGSDLLPAMGNHCSHVGGPQDLEK |
| | | TPRGRGSPLPPPREASLAVRGLLQELSSIR |
| | | HFLEKQDEMDEVAADWLRVGYVLDRLLFRI |
| | | YLLAVLAYSITLVTLWSIWHYS* |
| 10 | PSAM2 | ATGCGCTGTTCTCCAGGCGGCGTGTGGCTC |
| | nucleotide | GCCCTGGCTGCTTCCCTTCTGCACGTTAGC |
| | sequence | CTGCAGGGTGAGTTCCAGCGCAAACTGTAT |
| | | AAGGAGCTTGTTAAGAATTATAACCCCCTG |
| | | GAGCGGCCGGTCGCAAATGATTCCCAGCCA |
| | | CTGACAGTGTACTTCAGCCTCTCCTTGCTG |
| | | CAGATCATGGACGTGGATGAAAAGAACCAG |
| | | GTGCTGACCACTAATATTTGGTTGCAGATG |
| | | TCCTGGACCGATCACTACTTGCAGTGGAAT |
| | | GTGAGCGAATACCCAGGTGTAAAGACTGTA |
| | | AGATTCCCTGACGGCCAAATCTGGAAACCA |
| | | GATATCCTGCTGTACAACAGCGCAGACGAA |
| | | AGGTTTGATGCAACATTTCACACCAACGTG |
| | | GGAGTCAATTCTTCAGGCCACTGCCTGTAC |
| | | CTGCCCCCTGGAATCTTCAAGTCCTCATGC |
| | | TATATCGACGTCCGCTGGTTTCCCTTCGAC |
| | | GTCCAGCACTGCAAACTCAAATTCGGGAGC |
| | | TGGAGCTACGGCGGATGGAGCCTGGATCTG |
| | | CAAATGCAGGAGGCTGACATCTCTGGTTAC |
| | | ATCCCGAATGGGGAGTGGGACCTTGTGGGA |
| | | ATCCCCGGTAAAAGAAGCGAGCGATTTTAT |
| | | GAATGCTGCAAGGAACCCTTCCCTGACGTA |
| | | ACATTCACAGTTATCATCAGAAGAAGGCCA |
| | | TTGTTCTACGCCGTTAGTTTGTTGCTCCCC |
| | | AGTATTTTTCTCATGGTCGTGGACATCGTG |
| | | GGATTTTGTCTCCCACCTGATAGCGGGGAG |
| | | AGGGTCTCCTTTAAGATTACCTTGTTGCTC |
| | | GGCTATTCTGTATTTCTGATCATCGTGTCC |
| | | GATACCCTTCCTGCCACAATCGGCACTCCG |
| | | CTGATAGGAGTGTATTTCGTCGTGTGTATG |
| | | GCACTCCTGGTGATAAGTCTGGCGGAAACT |
| | | ATCTTCATTGTACGGCTGGTACATAAGCAG |
| | | GACCTGCAAAGACCCGTGCCAGACTGGTTG |
| | | CGACACCTTGTGCTGGACAGAATTGCATGG |
| | | ATTCTGTGTCTTGGCGAGCAACCTATGGCC |
| | | CACCGGCCACCTGCAACCTTTCAAGCCAAC |
| | | AAGACAGACGATTGTAGTGGGTCTGATCTG |
| | | TTGCCTGCTATGGGGAATCACTGCTCCCAT |
| | | GTTGGGGGACCACAAGATTTGGAAAAGACC |
| | | CCACGGGGGGGGGATCACCCCTTCCTCCTC |
| | | CCCGAGAAGCCTCTCTCGCTGTCCGGGGGC |
| | | TGCTCCAGGAACTGTCAAGCATCCGACATT |
| | | TTCTGGAGAAGCAGGACGAGATGGACGAAG |
| | | TCGCTGCCGACTGGCTGCGAGTGGGCTACG |
| | | TCCTTGACAGGCTGCTGTTTCGGATCTACT |
| | | TGCTGGCGGTGCTGGCTTATTCCATTACTC |
| | | TGGTGACACTCTGGTCCATATGGCACTACA |
| | | GTTAG |
| 11 | PSAM2 | MRCSPGGVWLALAASLLHVSLQGEFQRKLY |
| | protein | KELVKNYNPLERPVANDSQPLTVYFSLSLL |
| | sequence | QIMDVDEKNQVLTTNIWLQMSWTDHYLQWN |
| | | VSEYPGVKTVRFPDGQIWKPDILLYNSADE |
| | | RFDATFHTNVGVNSSGHCLYLPPGIFKSSC |
| | | YIDVRWFPFDVQHCKLKFGSWSYGGWSLDL |
| | | QMQEADISGYIPNGEWDLVGIPGKRSERFY |
| | | ECCKEPFPDVTFTVIIRRRPLFYAVSLLLP |
| | | SIFLMVVDIVGFCLPPDSGERVSFKITLLL |
| | | GYSVFLIIVSDTLPATIGTPLIGVYFVVCM |
| | | ALLVISLAETIFIVRLVHKQDLQRPVPDWL |
| | | RHLVLDRIAWILCLGEQPMAHRPPATFQA |
| | | NKTDDCSGSDLLPAMGNHCSHVGGPQDLEK |
| | | TPRGRGSPLPPPREASLAVRGLLQELSSIR |
| | | HFLEKRDEMREVARDWLRVGYVLDRLLFRI |
| | | YLLAVLAYSITLVTLWSIWHYS* |

### REFERENCES

1. Doane A S, Elemento O. Regulatory elements in molecular networks. Wiley Interdiscip Rev Syst Biol Med. 2017; 9(3):10.1002/wsbm.1374. doi:10.1002/wsbm.1374
2. Shlyueva, D., Stampfel, G. & Stark, A. Transcriptional enhancers: from properties to genome-wide predictions. Nat Rev Genet 15, 272-286 (2014). https://doi.org/10.1038/nrg3682
3. Latchman D S (December 1997). “Transcription factors: an overview”. The International Journal of Biochemistry & Cell Biology. 29 (12): 1305-12. doi:10.1016/S1357-2725(97)00085-X
4. Karin M (February 1990). “Too many transcription factors: positive and negative interactions”. The New Biologist. 2 (2): 126-31.
5. U.S. Pat. No. 9,950,068
6. U.S. Pat. No. 9,050,373
7. U.S. Pat. No. 9,314,529
8. U.S. Pat. No. 8,877,237
9. US Patent Application Publication No. 2020/0085974
10. US Patent Application Publication No. 2011/0274745

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A nucleic acid comprising a gene regulatory element sequence that has at least 85% identity to any one of SEQ ID NOs: 1-5, SEQ ID NOs: 17-22, or a fragment thereof.

2. The nucleic acid of claim 1, wherein the gene regulatory element sequence has at least 90%, 95%, 98%, or 99% identity to any one of SEQ ID NOs: 1-5, SEQ ID NOs: 17-22, or a fragment thereof.

3. The nucleic acid of claim 1 or 2, wherein the sequence comprises at least one modification relative to any one of SEQ ID NOs: 1-5 or SEQ ID NOs: 17-22.

4. The nucleic acid of any one of claim 3, wherein the modification is a substitution, an insertion, or a deletion.

5. The nucleic acid of any one of claims 1-4, further comprising a promoter.

6. The nucleic acid of any one of claims 1-5, further comprising a heterologous gene encoding a protein of interest.

7. The nucleic acid of any one of claims 1-6, wherein the gene regulatory element comprises any one of SEQ ID NOs: 1-5 or SEQ ID NOs: 17-22.

8. The nucleic acid any one of claims 1-7, wherein the gene regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides.

9. The nucleic acid of any one of claims 1-8, wherein the gene regulatory element comprises one or more transcription factor binding sites.

10. A nucleic acid comprising one or more gene regulatory elements comprising a nucleotide sequence at least 75% identical to any one of SEQ ID NOs: 1-5, or SEQ ID NOs: 17-22, and a nucleotide sequence encoding a protein of interest.

11. The nucleic acid of claim 10, wherein the one or more gene regulatory elements comprise one or more enhancers that enable selective expression of the protein in peptidergic and non-peptidergic nociceptors compared to other cells of the dorsal root ganglion (DRG) or compared to other cells of the central nervous system (CNS).

12. The nucleic acid of claim 10 or 11, wherein expression of the protein of interest is at least 10 times higher in peptidergic and non-peptidergic nociceptors than in other cells of the DRG or CNS.

13. The nucleic acid of any one of claims 10-12, wherein at least 80% of the DRG cells where the protein is expressed are peptidergic and non-peptidergic nociceptors.

14. The nucleic acid of any one of claims 10-13, wherein at least 90% of the DRG cells where the protein is expressed are peptidergic and non-peptidergic nociceptors.

15. The nucleic acid of any one of claims 10-14, wherein at least 95% of the DRG cells where the protein is expressed are peptidergic and non-peptidergic nociceptors.

16. The nucleic acid of any one of claims 10-15, wherein the one or more gene regulatory elements comprise a sequence that is at least 80% identical to any one of SEQ ID NOs: 1-5 or SEQ ID NOs: 17-22.

17. The nucleic acid of any one of claims 10-16, wherein the one or more gene regulatory elements comprise a sequence that is at least 90% identical to any one of SEQ ID NOs: 1-5 or SEQ ID NOs: 17-22.

18. The nucleic acid of any one of claims 10-17, wherein the one or more gene regulatory elements comprise a sequence that is at least 95% identical to any one of SEQ ID NOs: 1-5 or SEQ ID NOs: 17-22.

19. The nucleic acid of any one of claims 10-18, wherein the one or more gene regulatory elements comprise a sequence that is at least 98% identical to any one of SEQ ID NOs: 1-5 or SEQ ID NOs: 17-22.

20. The nucleic acid of any one of claims 10-19, wherein the one or more gene regulatory elements comprise a sequence that is identical to any one of SEQ ID NOs: 1-5 or SEQ ID NOs: 17-22.

21. The nucleic acid of any one of claims 1-20, wherein the one or more gene regulatory elements comprise binding sites for one or more transcription factors selected from the group consisting of Pit1 (POU Class 1 factors), Oct1 (POU Class 2 factors), HoxA11, GR, Arnt1, Srebf2, PU.1, and combinations thereof.

22. The nucleic acid of any one of claims 1-21 comprising two adeno-associated virus (AAV) inverted terminal repeats (ITRs), wherein the AAV ITRs are from a serotype selected from the group consisting of AAV1, AAV2, AAV3, AAV4, AAV5, AAV6, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, and AAV-PHP.eB.

23. The nucleic acid of any one of claims 1-22, wherein the nucleic acid comprises a promoter operably linked to a nucleotide sequence encoding the nucleotide sequence encoding the protein of interest and optionally a 5′ untranslated region (5′ UTR).

24. The nucleic acid of any one of claims 1-23, wherein the nucleic acid comprises one or more post-transcriptional regulatory elements.

25. The nucleic acid of claim 23 or 24, wherein the promoter is a beta globin promoter (pBG), a CAG promoter (pCAG), a AVIL promoter, a OPRM1 promoter, a TAC1 promoter, a SCN9A promoter, a SCN10A promoter, a Rbfox3 promoter, a RUNX1 promoter, a ISL1 promoter, a ISL2 promoter, a HMX1 promoter, a HOXD1 promoter, a CMV promoter, a human synapsin promoter, a chicken beta actin promoter, a PGK promoter, an Ef1a promoter, a ubiquitin promoter, a TATA-box containing promoter, a Calca promoter, a Taca promoter, a Trpv1 promoter, and variants thereof.

26. The nucleic acid of claim 24 or 25, wherein the post-transcriptional regulatory element is a Woodchuck Hepatitis Virus Posttranscriptional Regulatory Element (WPRE).

27. The nucleic acid of any one of claims 6-26, wherein the protein is a pro-neurogenic peptide, a pro-neurogenic protein, or an analgesic peptide.

28. The nucleic acid of any one of claims 6-26, wherein the protein is NGF, BDNF, NT-3, an opioid, a cannabinoid, or orexin.

29. The nucleic acid of any one of claims 6-26, wherein the protein is a membrane protein.

30. The nucleic acid of claim 29, wherein the membrane protein is an ion channel, a chemogenetic channel, an optogenetic channel, or a custom-designed chemogenetic/optogenetic channel.

31. The nucleic acid of claim 30, wherein the ion channel is an inhibitory ion channel.

32. The nucleic acid of claim 31, wherein the inhibitory ion channel a Kir2.1 inward-rectifier potassium ion channel.

33. The nucleic acid of claim 30, wherein the chemogenetic channel is an inhibitory chemogenetic channel, including Cation-Permeable Glycine Receptors, Ivermectin-activated Cation-Permeable Glycine Receptors, Pharmacologically Selective Actuator Modules (PSAMs), hM4Di and PSAM-GlyR.

34. The nucleic acid of claim 32, wherein the chemogenetic channel has a sequence that is at least 80% identical to any one of SEQ ID NOs: 7, 9 and 11, and is encoded by a nucleotide sequence having at least 80% identity with any one of SEQ ID NOs: 6, 8 and 10.

35. The nucleic acid of claim 32, wherein the chemogenetic channel has a sequence that is at least 90% identical to any one of SEQ ID NOs: 7, 9 and 11, and is encoded by a nucleotide sequence having at least 90% identity with any one of SEQ ID NOs: 6, 8 and 10.

36. The nucleic acid of claim 32, wherein the chemogenetic channel has a sequence that is at least 95% identical to any one of SEQ ID NOs: 7, 9 and 11, and is encoded by a nucleotide sequence having at least 95% identity with any one of SEQ ID NOs: 6, 8 and 10.

37. The nucleic acid of claim 32, wherein the chemogenetic channel has a sequence that is at least 98% identical to any one of SEQ ID NOs: 7, 9 and 11, and is encoded by a nucleotide sequence having at least 98% identity with any one of SEQ ID NOs: 6, 8 and 10.

38. The nucleic acid of claim 32, wherein the chemogenetic channel has a sequence that is at least 99% identical to any one of SEQ ID NOs: 7, 9 and 11, and is encoded by a nucleotide sequence having at least 99% identity with any one of SEQ ID NOs: 6, 8 and 10.

39. The nucleic acid of claim 32, wherein the chemogenetic channel has a sequence identical to any one of SEQ ID NOs: 7, 9 and 11, and is encoded by a sequence identical to any one of SEQ ID NOs: 6, 8 and 10.

40. The nucleic acid of any one of claims 1-38, wherein the nucleotide sequence further comprises a polyA signal.

41. A vector comprising the nucleic acid of any one of claims 1-39.

42. The vector of claim 40, wherein the vector is a viral vector.

43. The vector of claim 40, wherein the vector is a non-viral vector.

44. The vector of claim 41, wherein the viral vector is an AAV vector, a herpes vector or a lentiviral vector.

45. The vector of claim 43, wherein the viral vector is selected from the group consisting of an AAV1 vector, an AAV2 vector, an AAV3 vector, an AAV4 vector, an AAV5 vector, an AAV6 vector, an AAV7 vector, an AAV8 vector, an AAV9 vector, an AAV PhP-S vector, an AAV10 vector, an AAV11 vector, an AAVrh.10 vector, an AAV1 R6 vector, an AAV1 R7 vector, an rAAVrh.8 vector, an AAV-BR1 vector, an AAV-PHP.B vector, an AAV-PPS vector and an AAV-PHP.eB vector.

46. The vector of claim 42, wherein the non-viral vector is a plasmid.

47. A recombinant adeno-associated viral (rAAV) vector comprising a gene regulatory element sequence that has at least 85% identity to SEQ ID NOs: 1-5, SEQ ID NOs: 17-22, or a fragment thereof.

48. The rAAV vector of claim 46, wherein the gene regulatory element sequence has at least 90%, 95%, 98%, or 99% identity to SEQ ID NOs: 1-5 or SEQ ID NOs: 17-22.

49. The rAAV vector of claim 46 or 47, wherein the sequence comprises at least one modification relative to any one of SEQ ID NOs: 1-5 or SEQ ID NOs: 17-22, optionally a substitution.

50. The rAAV vector of any one of claims 46-48, further comprising a heterologous gene encoding a protein of interest.

51. The rAAV vector of claim 49, wherein the gene regulatory element comprises SEQ ID NOs: 1-5 or SEQ ID NOs: 17-22.

52. The rAAV vector of any one of claims 46-50, wherein the gene regulatory element comprises a sequence that is at least 500 nucleotides, at least 400 nucleotides, at least 350 nucleotides, at least 300 nucleotides, at least 250 nucleotides, at least 200 nucleotides, at least 150 nucleotides, at least 100 nucleotides, at least 50 nucleotides, or at least 25 nucleotides.

53. The rAAV vector of any one of claims 46-51, wherein the gene regulatory element comprises one or more transcription factor binding sites.

54. A recombinant adeno-associated virus (rAAV) comprising:
(i) a capsid protein from a serotype selected from the group consisting of AAV1, AAV2, AAV3, AAV4, AAV5, AAV6, AAV7, AAV8, AAV9, AAV10, AAV11, AAVrh.10, AAV1 R6, AAV1 R7, rAAVrh.8, AAV-BR1, AAV-PHP.S, AAV-PHP.B, AAV-PPS, and AAV-PHP.eB; and
(ii) the nucleic acid of any one of claims 1 to 39, or the rAAV vector of any one of claims 47-53.

55. A pharmaceutical composition comprising the nucleic acid of any one of claims 1-39, the vector of any one of claims 40-46, or the rAAV of any one of claims 47-53.

56. The pharmaceutical composition of claim 54, wherein the composition comprises lipid microparticle, a lipid nanoparticle, a polymeric microparticle, a polymeric nanoparticle, a liposome, an endosome, a micelle, a vesicle, a gold nanoparticle, a carbon nanotube, a quantum dot, a magnetic nanoparticle, a dendrimer, a calcium phosphate vehicle, a DNA-coated microparticle or a polymer.

57. The pharmaceutical composition of claim 54 or 55, wherein the composition further comprises a pharmaceutically acceptable carrier.

58. A method for selectively expressing a protein of interest in a peptidergic or non-peptidergic nociceptor, comprising contacting the nociceptor with the nucleic acid of any one of claims 1-39, the vector of any one of claims 40-46, the rAAV vector of any one of claims 47-53, the rAAV of claim 54, or the pharmaceutical composition of any one of claims 55-57.

59. A method of treating a neurological disease or condition in a subject comprising administering to the subject the nucleic acid of any one of claims 1-39, the vector of any one of claims 40-46, the rAAV vector of any one of claims 47-53, the rAAV of claim 54, or the pharmaceutical composition of any one of claims 55-57.

60. The method of claim 59, wherein the subject is a non-human mammal.

61. The method of claim 59, wherein the subject is a human.

62. The method of any one of claims 59-61, wherein the administering to a subject is through injection.

63. The method of claim 62, wherein the injection comprises intracerebroventricular (ICV), intravenous (IV) injection, or injection into the cisterna magna (ICM).

64. The method of any one of claims 59-63, wherein the administering results in a reduction of abnormal activity of nociceptors.

65. The method of any one of claims 59-64, wherein the neurological disease or condition is pain, back pain, chronic pain, a peripheral neuropathy, a diabetic neuropathy, neuralgia, a chemotherapy-induced neuropathy, migraine, or trigeminal neuralgia.

66. The nucleic acid of any one of claims 1-39, the vector of any one of claims 40-46, the AAV vector of any one of claims 47-53, the rAAV of claim 54, or the pharmaceutical composition of any one of claims 55-57, for use in the treatment and/or management of pain or a neurological disease.

67. A kit for use in the treatment and/or management of pain or a neurological disease, comprising the nucleic acid of any one of claims 1-39, the vector of any one of claims 40-46, the AAV vector of any one of claims 47-53, the rAAV of claim 54, or the pharmaceutical composition of any one of claims 55-57, and instructions for preparation and mixing a sample and administering it to a subject.
